(19) United States
(12) Patent Application Publication
(10) Pub. No.: US 2011/0008775 A1
(43) Pub. Date: Jan. 13, 2011

(54) SEQUENCING OF NUCLEIC ACIDS

(76) Inventors: Xiaolian Gao, Houston, TX (US); Xiaochuan Zhou, Houston, TX (US)

Correspondence Address: G Kenneth Smith, 1645 Briarwood Circle, Bethlehem, PA, 18015, US

(21) Appl. No.: 12/735,009

(22) PCT Filed: Dec. 10, 2008
(86) PCT No.: PCT/US2008/086302

§ 371 Date: Sep. 22, 2010

Related U.S. Application Data
(60) Provisional application No. 61/012,468, filed on Dec. 10, 2007.

(51) Int. Cl. C12Q 1/68; C12P 19/34; C12M 1/34

(52) U.S. Cl. 435/6; 435/91.5; 435/287.2

(57) ABSTRACT

The present invention relates to the field of analysis of nucleic acid sequences. More specifically, the present invention relates to the method and instrument for high throughput parallel DNA sequencing. The present invention also provides method for selection of sequences from analyte samples for enrichment of the target sequences or depletion of the selected molecules and in particular undesirable sequence templates from sequencing samples.

### CROSS REFERENCE TO RELATED APPLICATIONS

This application claims priority to the filing date of U.S. Provisional Application No. 61/012,468 filed Dec. 10, 2007; the disclosure of which is herein incorporated by reference.

### BACKGROUND OF THE INVENTION

1. Field of the Invention

The present invention relates to the field of analysis of nucleic acid sequences. More specifically, the present invention relates to the method and instrument for high throughput parallel DNA sequencing. The present invention also provides method for selection of sequences from analyte samples for enrichment of the target sequences or depletion of the selected molecules and in particular undesirable sequence templates from sequencing samples.

2. Description of the Prior Art

Genomic DNA provides the code for basic biological systems and transcriptome RNA provides the footprint for proteins and other RNA elements whose functions are of scientific interest. The field of DNA/RNA sequencing is of fundamental importance to deciphering these systems and thus has experienced exponential growth over the past few decades. The genesis of several next generation sequencing technologies have recently stimulated excitement in not only megabase throughput but also broad applications relating to genomes and transcriptomes, such as rapid complete genome sequencing and re-sequencing, SNP detection, long DNA genetic mutation analysis (epigenetic analysis), detection and profiling of small RNA, ncRNA, protein and biologically important RNA molecules, fueling the fields of genomics and metagenomics. These deeper and more comprehensive genetic and transcriptome analyses can be applied in basic research (function identification, pathway construction, interaction mapping, systems biology, ecological evolution, disease mechanistic studies, etc.) as well as applied clinical fields (biomarkers for disease early detection, prediction, prevention and treatment). In domains usually occupied by microarrays, sequencing is increasingly used.

The Sanger sequencing method^{Error Bookmark! Bookmark not defined.} (Sanger et al. (1975) J. Mol. Biol. 94, 441-448; Sanger et al. (1977) Proc. Natl. Acad. Sci. USA 74, 5463-5467; Sanger et al. (1977) Nature 265, 687-695; Maxam et al. (1977) Proc. Natl. Acad. Sci. USA 74, 560-564; Szekely et al. (1977) Nature 267, 104) has been the major workhorse behind human genome sequencing (Lander et al. (2001) Nature 409, 860-921; Venter et al. (2001) Science 291, 1304-1351; International Human Genome Sequencing Consortium (2004) Nature 431, 931-945; Levy et al. (2007) PLoS Biol 5, e254), owing to its advantages of longer reads (700 bp of routine read length), higher accuracy (99.0% on a single pass), and its simple process and reliability over other sequencing methods. This process involves preparation of the sample, often a PCR product or an amplicon; the amplicon is then taken through a sequencing reaction such as AB's BigDye terminator cycle reaction, in which the DNA polymerase incorporates regular dNTP and a small portion (˜1%) of 2′,3′-dideoxy-ddNTP terminators to extend the chain length by base pair recognition. These activities result in a series of chain-termination DNA fragments different in lengths by one nucleotide and the fragments are fluorescence-labeled through termination fluorescent dye incorporation. The sequence mix is resolved using time slab or, later, capillary electrophoresis analysis and the reads in single base staggered lengths are detected using orthogonal laser or light irradiation into the row of capillaries, and signals are acquired by photomultiplier or CCD devices, and shown in chromatogram graphs to produce base calls. Very long reads (1,000-1,300 bp) were reported at a low error rate (accuracy>99%) and in only one to two hours using linear polyacrylamide composition mixtures at elevated temperatures and optimized electric field (Zhou et al. (2000) Anal Chem 72, 1045-1052; Carrilho et al. (1996) Anal Chem 68, 3305-3313). In automated sequencers, one round of AB's (Applied BioSystems) 96-channel capillary electrophoresis (CE) analysis generates a total of 96×700, or 67.2 Kbp base pair reads per run (Table 1). Human genome/large scale sequencing has been accomplished by employment of an army of AB's sequencers by a few national human genome sequence centers such as the Baylor Human Genome Sequencing Center and the Washington University Genome Sequence Center; the instrument costs $350K each (AB 3730xl 96 Capillary Sequencer).

The automated ABI capillary electrophoresis sequencer has 1D capillary array consisting of 8, 24 or 96 capillary channels. The detection is from the side of the array. 1D array has limitation in the number of samples can be analyzed. WO 2007/084702 describes methods and devices used in the sequencing and separation, detection and identification of biological molecules. As to DNA sequencing the specification describes a system based on cyclic sequencing by synthesis which is performed on beads in three dimensional vessels and detected using monolithic capillary arrays. The specification describes the use of quantum dots and multiple luminescent labels for detection. The specification describes detection of fluorescent signals from beads pumped through tubes a monolithic multi-capillary array. Detection of individual beads is done from the top of the array in real time fashion using lasers or LEDs as illumination sources and fast CCD cameras as detection.

There has been continued effort in miniaturizing and integrating the devices for PCR, sample purification, capillary electrophoresis, and signal detection (Dolnik et al. (2000) Electrophoresis 21, 41-54; Liu et al. (2000) Proc Natl Acad Sci USA 97, 5369-5374; Blazej et al. (2006) Proc Natl Acad Sci USA 103, 7240-7245; Liu et al. (2006) Anal Chem 78, 5474-5479; Blazej et al. (2007) Anal Chem 79, 4499-4506; Kumaresan et al. (2008) Anal Chem 80, 3522-3529; Liu et al. (2007) Anal Chem 79, 1881-1889). Notably microfabricated chips in their optimal settings can rapidly (in minutes to 1-2 hours) detect DNA fragments of 300 bps to kbp lengths at attomolar sensitivities. A portable PCR-CE device used in a test case of four amplicon samples achieved detection of 20 copies of DNA^{14e}. While it is encouraging that the traditional CE can be further miniaturized and sensitivity can be further improved, in the race to increase the sequencing capacity, Sanger sequencing runs into a bottleneck for lacking a vehicle to embrace the needs of gigabase sequencing. The major workhorse behind genome sequencing has been the Sanger sequencing method. This process involves preparation of the sample, often a PCR product or amplicon. The amplicon is then subjected to a sequencing reaction (i.e. ABI's BigDye terminator cycle reaction) in which DNA polymerase incorporates 2′,3′-dideoxy-dNTP terminators to produce early chain termination DNA fragments. The reaction mix is analyzed using electrophoresis analysis and the sequencing results are shown in chromatogram graphs. In automated sequencers, one round of ABI's96-channel capillary electrophoresis (CE) analysis generates a total of 96×700, or 67 kilobase (kbp) reads. Genome/large scale sequencing has been accomplished by employing an army of sequencers, costing $350K each (ABI 3730xl 96 Capillary Sequencer). In CE sequencing, the sequencing samples are prepared for each sequence and subsequently loaded onto a 96 well plate. CE on microchips, promising faster and easier results, has been reported, but the mode of operation is fundamentally similar to that of the automated CE sequencer. A genome sequencing task using conventional methods would require million dollar facility set up, days of time, and $5 M-$10 M in material costs. Additionally, as the number of sequences to be analyzed increases, the number of PCR and fluorescence terminator sequence reactions increases. Robotics and sample handling/storage required to handle the large numbers of reactions become costly. Clearly, in order to address the many applications of DNA analysis, it is necessary to continue to significantly reduce the cost of sequencing and increase the speed of reading DNA sequences by technology advancement.

Pyrosequencing has been described in various publications and patent including U.S. Pat. Nos. 6,210,891, 7,264,929 and 7,335,762. In pyrosequencing templates are prepared by emulsion PCR with one to two million beads deposited into PTP wells. Smaller beads with sulphurylase and luciferase attached thereto surround the template beads and individual deoxynucleotide phosphates (dNTPs) are sequentially dispensed over the across the wells. When a dNTP which is complementary to the template is incorporated into the growing strand a pyrophosphate (ppᵢ) is released and converted to ATP. The ATP oxidizes the luciferin to oxyluciferin and light is released. A detector detected the light released and correlates that event with the dNTP incorporated. This technique provides for reads of about 400 bases and can detect a homopolymer string of around six bases. The technique is susceptible to insertion and deletion errors.

Sequencing by ligation has been described in various publications and patents including U.S. Pat. Nos. 5,912,148 and 6,130,073. In sequencing by ligation around one hundred million emulsion PCR template beads are deposited onto a glass slide and a universal primer is annealed to the templates. Probes containing two interrogation bases, each set of interrogation bases having a selected dye associated with it are added to the templates and those complementary to the target sequence are annealed. The 16 different dinucleotides within the probes are encoded in 4 different dyes. Following four color imaging the ligated dinucleotide probes are chemically cleaved to generate a phosphate group. The cycle of hybridization, ligation, imaging and cleaving is repeat a total of seven times so that the correct two base sequence can be identified. Next the universal primer is removed from the template and a second ligation round is performed with an n−1 primer which sets the interrogation base one base to the 5′ end. Seven more rounds of hybridization, ligation, imaging and cleaving are performed and 3 more rounds of removal and ligation produces a string of 35 data bits encoded in color space. These are aligned to a reference genome to decode the DNA sequence. This technique is limited by the short run length, 35 bases, and is prone to substitution error.

There are two techniques that employ reversible terminators to accomplish DNA sequencing. In the first, bridge amplification of DNA fragments is randomly distributed across eight channels of a glass slide, to which high density forward and reverse primers are covalently attached. The solid phase amplification produces about total 80 million molecular clusters from individual single strand templates. A primer is annealed to the free ends of templates in each molecular cluster. The polymerase extends and then terminates DNA synthesis from a set of four reversible terminators each labeled with a different dye. Unincorporated reversible terminators are washed away and base identification is done with four color imaging. Blocking and dye groups are removed by chemical cleavage so that another cycle can be performed. This technique is limited by the short run length, 35 bases, and is prone to substitution error.

In the second technique using reversible terminators billions of unamplified ssDNA templates are prepared with poly(dA) tails that hybridize to poly(dT) primers covalently attached to a glass slide. For one pass sequencing this primer-template complex is sufficient, but for two pass sequencing the template strand is copied, the original template is removed and annealing a primer directed toward the surface. Unlike the first reversible terminator technique the reversible terminators are all labeled with the same dye and dispensed individually in a predetermined order. An incorporation event results in a fluorescent signal. U.S. Pat. No. 7,169,560 describes methods utilizing this reversible primer technology. If single molecules are not used then de-phasing, where thousands of copied templates within a given molecular cluster do not extend their primers efficiently, are not extended can be a problem. This technique is limited by the short run length, 25 bases, and is prone to deletion error.

Sequencing by fluorescence resonance energy transfer (FRET) signal generated during the incorporation, by DNA polymerase labeled with a FRET molecule, of a cognate dNTP labeled with a FRET molecule at its terminal phosphate group. The labeled dNTP is incorporated when it has the correct complementary to the template strand and the FRET due to the interaction of the two FRET molecules marks the base extension event, giving rise to the sequence read. This method has the advantages in recording DNA polymerization in real time and regular DNA without any modification is synthesized, and thus longer DNA reads can be recorded Us patent applications [Hardin, et al. U.S. Pat. No. 7,329,492; Korlach, et al. U.S. Pat. No. 7,361,466] and in literature by Eid, J. et al. (2008) [PMID: 19023044]. These methods have not been demonstrated for sequencing the full base content of a DNA molecule.

Toward these ends of increased speed and decreased cost developments including sequencing DNA by hybridization, by synthesis (3′-extension), by ligation, by polony polymerization, by nanopore, by polymerase incorporation of dye-labeled dNTPs, and a few others have been developed. The rapid progress in DNA sequencing technologies (e.g. 454′s high throughput pyrosequencing (454 Life Sciences) (Margulies et al. (2005) Nature 437, 376-380; Wheeler et al. (2008) Nature 452, 872-826; Ronaghi, et al. (1996) Anal Biochem 242, 84-89; Ronaghi et al. (1998) Science 281, 363-365), Illumina/Solexa sequencing by synthesis from single clones on a surface (Illumina) (Margulies et al. (2005) Nature 437, 376-380; Wheeler et al. (2008) Nature 452, 872-826), ABI's SOLiD technology (“Supported Oligonucleotide Ligation and Detection”, Applied Biosystems)) (Cloonan et al. (2008) Nat Methods 5, 613-619), genomics assays, and bioinformatics technologies have dramatically opened up the opportunities for researchers to obtain in depth molecular pictures of complex biological systems.

Technologically, the next generation sequencing technologies simplify and accelerate sequencing by a) eliminating the need for individual cloning in sample preparation as required in traditional sequencing; b) parallel preparation of millions of sequences to be analyzed, and c) simultaneously detecting sequencing signals in millions of events. However, this generation of large scale sequencing technologies suffers from a few common shortcomings, which include: d) All are stepwise (cyclic) reactions for each addition of dNTP and this inherently limits total length of the sequencing methodology (Table 1 and Solexa and SOLiD sequencing length will not be possible to exceed 100bp). e) The cyclic reactions also limit the speed of full length sequencing. Solexa sequencing takes more than about two hours at each step and overall 35 nucleotide additions require 2 days or more, and SOLiD takes twice as long time. f) Some approaches require modification of dNTP and these modifications further increase the cost and introduce other issues such as material stability during storage and use. g) 454 cannot resolve repeat sequences in genome. h) The quality of the sequencing reads is very poor towards the later 10% of the sequence. i) Deep sequencing is required for de novo sequencing using short reads, up to 20× can be possible. In particular the current technology provides insufficient base-read length. The base-read lengths for the current next-generation sequencing methods are too short to be robust for assembling the final long DNA with sufficiently high accuracy for re-sequencing and/or for de novo sequencing of new genomes. A stretch of DNA of 20-30 nucleotides may occur multiple times in a genome, and therefore there are ambiguities as to their counts as the abundance copies or as multiple presences in the genome. In addition, some genomes, such as human, are full of repeating sequences, and in these cases, the sequencing base-read lengths of ˜30 bps leave their precise genomic location uncertain. It is highly desirable to enhance the ability of the new ultra-fast sequencing technologies so that the base-read length is at least comparable to or higher than that obtained by the conventional sequencing methods, such as Sanger sequencing. One can imagine that such sequencing technology will greatly expand the range and scope of sequencing applications to those requiring more reliable quantitative measurements of DNA or RNA copies, those of measurements relying on longer sequence information such as new genome sequencing and highly mutable or trans-splicing coding sequence studies. Such progress in technology would also reduce the time needed for data analysis, adding the benefit of time-saving and/or an increase in overall throughput.

Therefore, even with the progress outlined above there are several areas to be improved in these technologies if the full potential of DNA sequence analysis in human healthcare and basic life science research is to be realized.

Second, improvements in target-specific sequencing are also required. The new sequencing methods described above randomly pick up sequencing amplicons and thus have limited access to the entire population (e.g. in case of 25/48 barcode sequences were detected in 454 pyrosequencing (Leamon et al. (2007) Gene Ther. Reg. 3, 15-31) and the representation would decrease with samples of a larger population and low abundant populations and the methods could suffer from selection bias due to natural or experimental preference for certain kinds of sequences). Pyrrosequencing depends on the intensities of the Therefore, the prior art methods may be suitable for discovery but are not a substitute for the conventional target-specific Sanger sequencing as there is no guarantee that a specific sequence will definitely be sequenced and multiple passes (usually 10×-20×) of the sequencing runs are required to ensure a reasonably complete coverage of target sequences and sequencing accuracy. This sampling limitation excludes many applications since DNA is full of repeats and functionally unknown sequences. In addition, the region of interest varies widely with each research question, for instance, regions of coding or non-coding sequences (small RNA, intronic, intergenic, untranslated), SNP, regulatory regions (replication, transcription and/or translation regulation, other genetic function regulation), areas of imprinting/methylation, trans-spliced and transposon regions, or any combination of these. DNAs of different organelles may also be selected. There are also many existing biomedical genomic applications, such as clinical assays, which are likely to look at a small set of genes or mutation sites but need to cover a large set of samples. Given these needs and the still considerably high cost per run for these next-generation sequencing technologies, it is highly desirable that the ultra-fast methods can be applied for target-specific sequencing to allow a high number of different samples to be analyzed and systematically studied per reaction run. Overcoming the current sampling limitation will be a tremendous step forward in fully realizing the potential of the sequencing technologies for general research as well as clinical laboratory applications.

Finally, the processes of the next generation sequencing technologies need to be simplified. The sample preparation and/or sequencing processes are presently cumbersome, requiring several days and involving multiple steps of enzymatic reactions, sequence-extension by synthesis and four-base cycles per chain-length extension. These complicated procedures tend to be associated with unstable results, cause experimental failures, demand technical expertise, and lengthen experimental time. The present invention provides a robust system for sequencing that is highly automated and can be routinely used to generate megabase (Mbp) to Gbp data. The methods of the present invention have advantages compared with the methods such as sequencing by synthesis in the new era of next-generation sequencing. The present invention can eliminate the need for individual sample preparation normally required for conventional sequencing, and significantly increases the throughput of target-specific sequencing at a rate comparable to the next-generation sequencing methods. The devices and methods of the present invention will also generate long and more accurate reads that are comparable to conventional sequencing methods while providing many more simultaneous reads thereby increasing throughput over conventional sequencing by thousand folds.

For large scale experiments, in many cases one would desire to select for smaller subsets, which can be done for nucleic acids by hybridization. Separations are usually done by chromatography (affinity separation, separation by physical separation such as precipitation and liquid layer separation), and increasing by beads. These are small particles, porous and nonporous, of the various shapes (disk, sphere, rod, square, etc.), hollow or solid or in layers or with a core and shell, made from a variety of materials including but not limited to glass, ceramic, polymer, metal, metal ion, semiconductor, and combination of more than one material. For example, a bead may contain a paramagnetic core encapsulated or coated with film of polymer material. The paramagnetic core facilitates transportation, sorting, and holding of the bead using magnetic force. Another exemplary bead contains a paramagnetic coating, at least on one or more sections of the bead, also to facilitate bead manipulation by magnetic force. Yet another exemplary bead contains a solid core, such as glass, that is encapsulated with a layer of polymer matrix material for increasing synthesis load. The matrix material includes but is not limited to low cross-linked polystyrene, polyethylene-glycol, and various copolymer derivatives

The surface of beads can carry functional groups and molecules, such as primers for nucleic acid amplification using PCR, isothermal amplification, rolling circle amplification, and other methods to multiply the copies of nucleic acids, i.e. DNA or RNA. The surface molecules can also carry specific hybridization probes, which are capture probes or captors for retaining sequences on surface for future applications. The beads carry primers, captors, and other types of oligonucleotides are called probe beads.

Although oligonucleotide synthesis on beads is carried out routinely in commercial places and research laboratories, the synthesis on a pico-liter scale can only be carried out using a pico-liter array chip device to reach parallel synthesis of thousands and more of different, pre-designed oligos in up to fmol quantities of each sequence (Tian et al. (2004) Nature 432, 1050-1054; Zhou et al. (2004) Nucleic Acids Res. 32, 5409-5417). Oligonucleotides and their modification derivatives are modified analogs which can improve the properties required for applications. The synthesis capability and the availability of the various beads are methods possible for creating probe beads for selection of sequencing targets as described in PCT/US08/82167.

The various methods are developed for probe design based on nucleic acid complementary strands interact to form base pairs and helical structures. Hybridization specificity and affinity are important parameters for evaluation of the probes. There are also different functions for which probes are designed. One kind of probes is designed to be highly specific for a single target and there should be no-cross hybridization present. Another kind of probes is for capture a region or a few regions in the target sequence, such as a 10 Mbp susceptible cancer gene genomic region. Probes for capturing such as region can be designed using strategies differing, for instance, in considerations of specificity, the length of the target sequences, and the distribution densities (probes per number of base pairs). Therefore, besides the highly sequence specific probes, the second type of probes may be those of tiling, i.e. probes are overlapping and sequentially shifted by one or more nucleotides. Such probes are redundant, heterogeneous in their hybridization specificity and affinity (most times expressed as Tₘ, melting point). The nature of the capture by such kind probes is essentially random and the copies of the captured sequences will be largely different. This also means the copies of the target sequences may vary greatly. The third type probes are designed over a region and probes are separated by evenly distributed over the interested target region. The distance (measured in nts) is determined by the average length of the sample sequences. For instance, the distance from probe to probe is about the same as the average length of the target sequences (assuming the target sequences are random fragmentation product). In this case, probes can be selected in the small region with 2-3 probe length with better properties. Overall, the probes of this kind have better efficiency in hybridization and quality. Each target sequence should match at least one probe.

In the probe application for selecting target sequences, it may be desirable to reduce the number of probes and to have a minimal set of probes to hybridize with the target sequences, where one probe is purposely designed to hybridize with as many target sequences as possible in a consensus sequence (CR) region (FIG. 20). This is illustrated in FIG. 10, it shows 10 lines (S1, S2, . . . to S10) representing 10 DNA sequences and three CR probes (CR1, CR2, CR3). Normally, 10 probes will be required; but shown in FIG. 20, CR1 probe captures three target sequences (FIG. 21, where at the mismatch position, the synthesis of the CR1 probe incorporates a mixture of A, C, and G and thus in fact the specific probes for the three targets are synthesized), CR2 captures four targets, and CR3 capture five targets. Targets S3 and S10 are captured twice. Following the working principle, there are applications highly specific hybridization is not necessary, it is possible to allow the presence of mismatches and thus expand the number of different targets for a single CR probe. This consensus probe hybridization strategy will save the cost of probe synthesis and since the target sequences are hybridizing to the same probe and thus the relative copies of hybridization of the different target sequences are the about the same. Therefore, CR probes also reduce the differential in hybridization copy numbers. FIG. 22 shows the CR probes can be immobilize to beads to facilitate the use. In one preferred embodiment of the present invention, the bead is streptavidin coated and CR probes are modified with biotin. The CR probes on magnetic beads are hybridized with the targets and the beads are washed to elude de-selected sequences. The hybridized target sequences are eluded and collected for next step application. A useful applications of the CR probes and the specific capture probes are to enrich target sequences for miRNAs and CR probes are mature miRNA complementary sequences. Other examples include cancer genes, P450 genes, HLA genes, etc. The target sequences are obtained from sequence databases and analyzed by alignment based on a set of selection rules (such as mismatches allowed) to identify CR probes.

### SUMMARY OF THE INVENTION

The present invention relates to devices and methods for high-throughput, long-read, accurate, fast, and low-cost sequencing of DNA. The present invention relates to a next generation long-read sequencing (NG-SS, Next Generation Sanger Sequencing) technology, which utilizes the advantages of time-proven Sanger sequencing and capillary electrophoresis to establish a new platform that will perform microbead-based Sanger sequencing reactions in a massively parallel scale, by separately placing millions of different sequences in a three dimensional (3D) high density capillary module, electrophoretically separate sequencing fragments, rapidly acquiring fluorescence images on the exit plane of the capillary module, and using the rapidly recorded time-resolved images to re-construct sequence information. The combination of these approaches provides reliable methods which overcome the short-read and stepwise (or cyclic) reaction limitations in all of the present next generation sequencing methods. The methods and devices of the present invention increase the throughput of the conventional Sanger sequencing method thousands fold. The device of the present invention provide sequencing instruments that are simple and fast to operate, capable of high accuracy reading genome-scale sequences (billion bps) in hours and at a cost of less than these presently available devices and methods.

In addition to the long read, the devices and methods of the present invention present advancement over the prior art in that high throughput sample processing will obviate cloning. The devices of the present invention utilize a 2D capillary module rather than 1D capillary tube alignment, thereby increasing throughput n times (n being the number of rows in the second dimension). The devices of the present invention provide for millions of sequencing capillaries The methods of the present invention provide high capacity short target sequences may be linked together into a continuous polymer (i.e. concatemers) and provide more accurate sequencing especially for homolog stretches, long repeats, and structure variation sites. The methods of the present invention significantly reduced sequencing time, as there are no dNTP-sequencing stepwise cycles as now used in all three current next generation sequencing methods (454 sequencing needs pyrophosphate detection and adding one kind of dNTP at one time, Solexa sequencing requires addition of dye labeled dNTP each cycle, and SOLiD sequencing needs 5 sets of ligation oligos for each reaction run). In the methods of the present invention sequencing data of each capillary channel can be continuously recorded. The methods and devices of the present invention significantly reduce sequencing redundancy requirements (e.g. Solexa and SOLiD sequence require about 20× redundancy for genome sequencing); therefore the methods of the present invention produce savings in time and cost for re-sequencing. The present invention provides capillary electrophoresis (CE) array modules that are reusable many times after flush out the filling gel. No molecules are derived on capillary surface and thus the CE block is renewable. The CE devices of the present invention can be modular and it is possible to build a small laboratory or a genome sequencer for addressing both the genomic scale and routine sequencing needs. The present invention provides devices and methods for simultaneous sequencing and parallel nucleic acid copy measurements by target-specific capture of the analyte sequences. The measurements can be in very large scales which will be far exceeding the current 300 nanoliter reaction plate from Biotrove; and with the sequence information, the method minimizes false positives compared to the current probe-based real-time PCR measurements where sequences are only recognized by hybridization. The ultra-fast sequencing and the hybridization microarray will be complementary technologies for discovery as well as comprehensive, in-depth, accurate and quantitative analyses of DNA and RNA from samples of genome-scale or small specific subsets.

### BRIEF DESCRIPTION OF THE DRAWINGS

FIG. 1 is a schematic drawing of a pico-liter microfluidic array synthesis device.

FIG. 2 is a planar glass plate for array synthesis.

FIG. 3 is a schematic drawing of a binary bead sorting synthesis system.

FIG. 4 is a schematic drawing of an exemplary bead synthesis system and process.

FIG. 5 is a schematic illustration of one embodiment of an oligo probe bead molecule.

FIG. 6 is an illustration of a synthetic probe synthesized on surface.

FIG. 7 is a microscope image of a reaction chamber filled with reaction beads.

FIG. 8 is an illustration of probe beads as amplification primers.

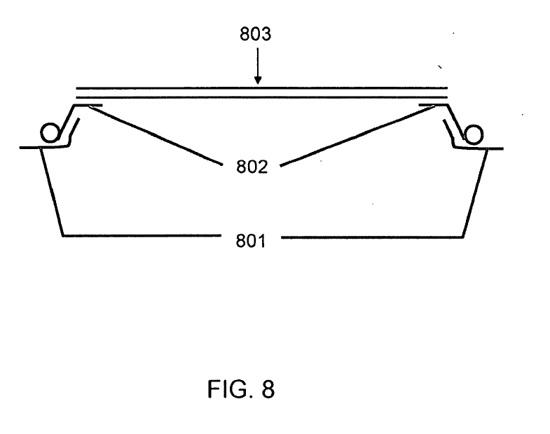

FIG. 9 is an image of beads on surface.

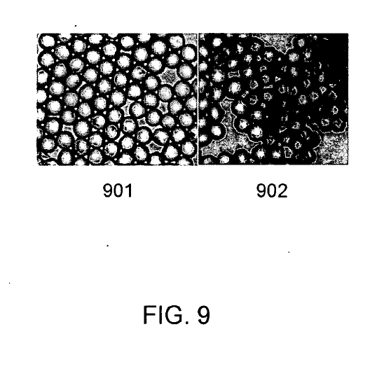

FIG. 10 is an experimental flow comparing the results of using or without using magnetic streptavidin bead for oligo mixture processing.

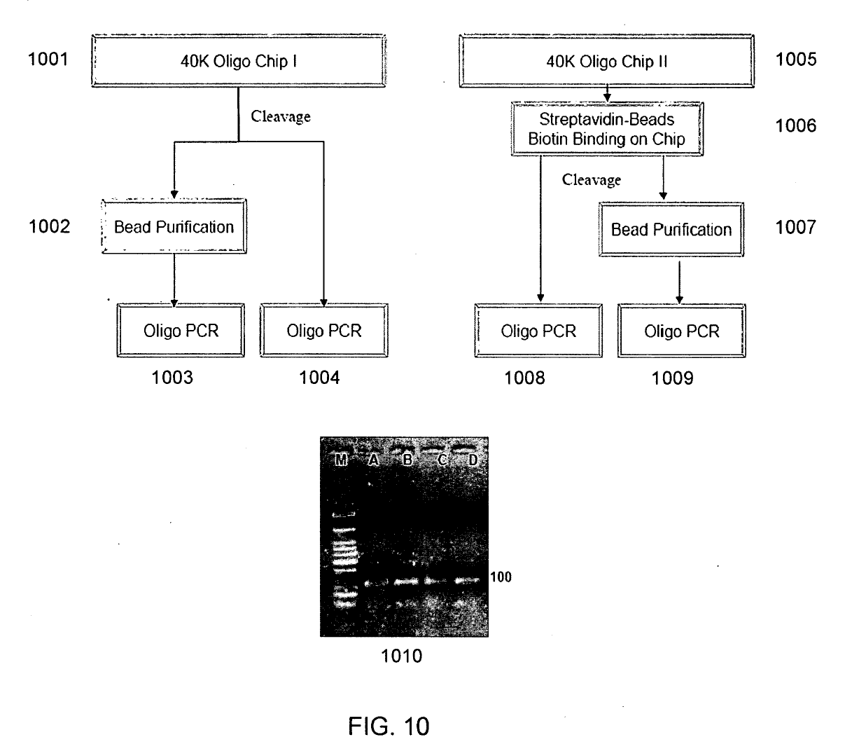

FIG. 11 is a schematic illustration of the one of the methods of sample preparation of the present invention. Steps 1111 to 1114 are designed to perform clonal emPCR amplification of single DNA molecules to produce beads with each containing amplicons of a single sequence. Steps 1115 to 1118 are designed to perform Sanger reaction to produce, on each bead, a full set of cleaned, fluorescence-labeled Sanger sequencing fragments of a single template sequence.

FIG. 12 is a schematic illustration of emulsion Sanger amplification reaction on a bead attached with a Sanger product capture sequences.

FIG. 13 is a schematic diagram of an integrated system consisting of a capillary array electrophoresis subsystem and a fast confocal laser scanning microscope detection subsystem.

FIG. 14A is a cross-section view of schematic diagram of an electrolyte cell using capillary array.

FIG. 14B is a 3D illustration of a capillary array block.

FIG. 15 schematically shows an enlarged portion of source-chamber end of a capillary cell.

FIG. 16 is a schematic illustration of the process from image data to sequence.

FIG. 17 is images detected over the time course of DNA gel migration and the time-dependent signal intensities are sketched on top of the images from two emission wavelength (FAM: 510 nm and Cy3: 535 nm).

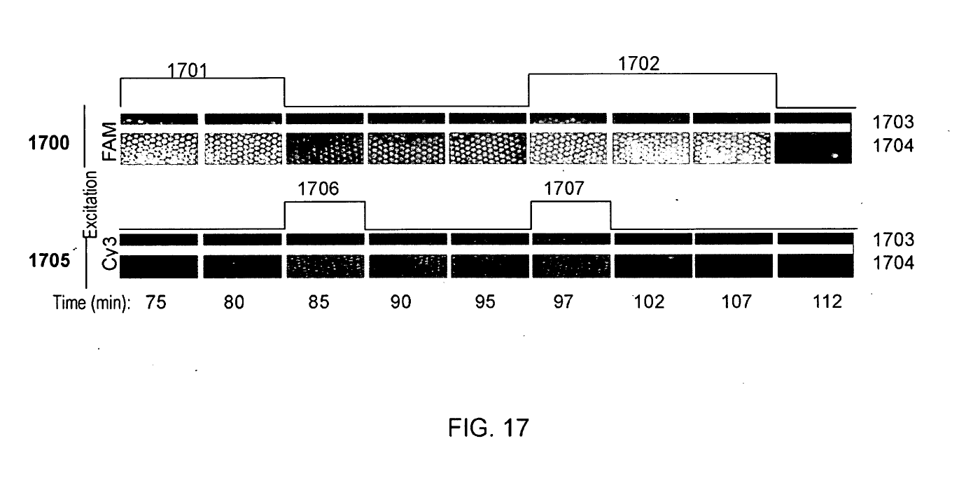

FIG. 18 is a time-resolved image taken from side, perpendicular to the capillary channels.

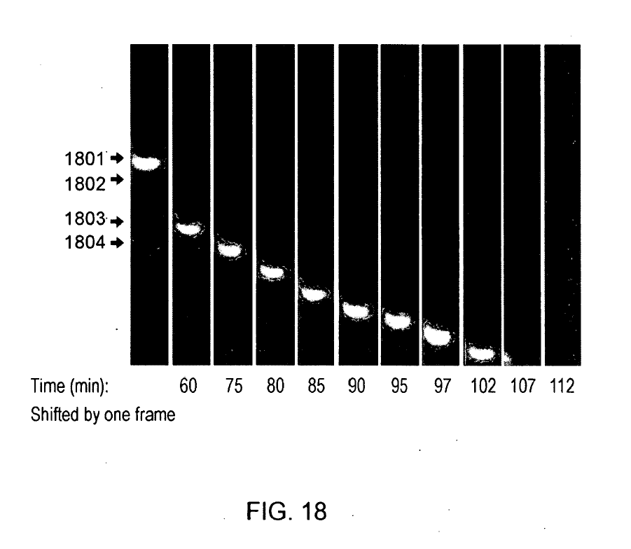

FIG. 19 is an enlarged view of the microfabricated capillary chip surface showing beads were loaded into capillary channels filled with gel.

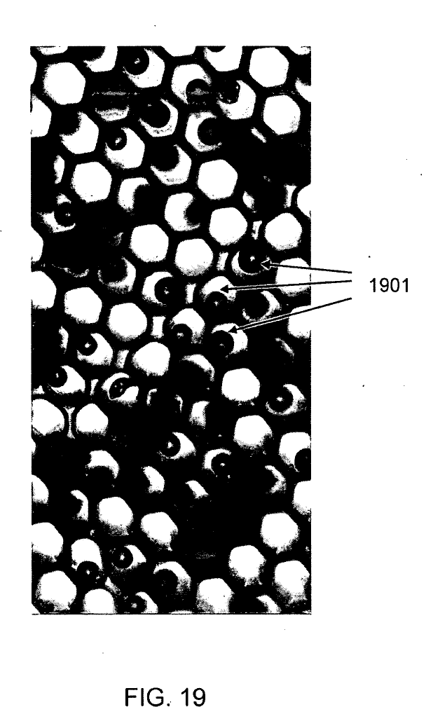

FIG. 20 is a schematic illustration of finding consensus regions (CR) for a set of DNA or RNA sequences (e.g. S1, S2 . . . S10, but these are not limited to 10 sequences).

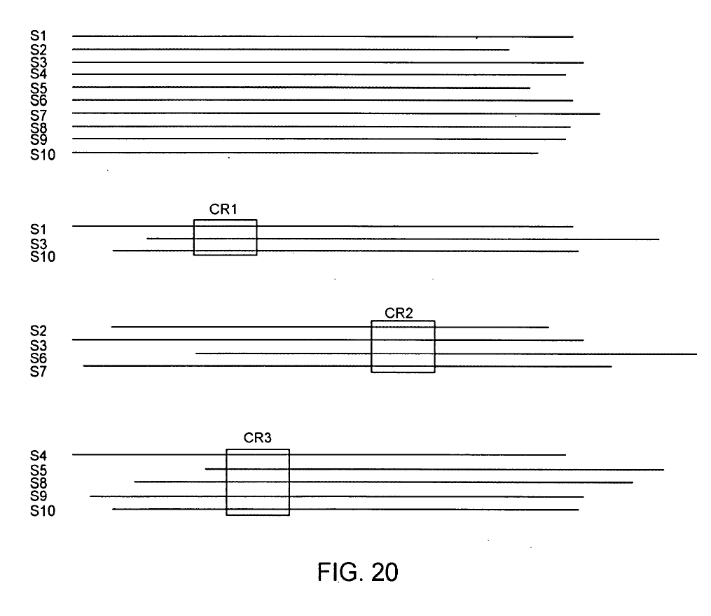

FIG. 21 is a schematic illustration of designing a consensus region (CR) probe for a set of DNA or RNAs.

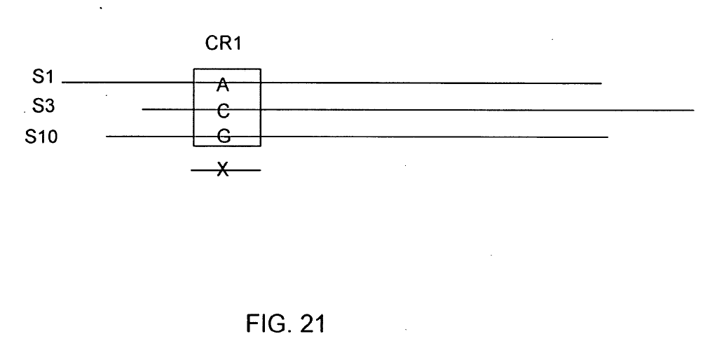

FIG. 22 is a schematic illustration of using CR probe on magnetic bead (but not limited to magnetic bead) to capture target DNA or RNA sequences by hybridization.

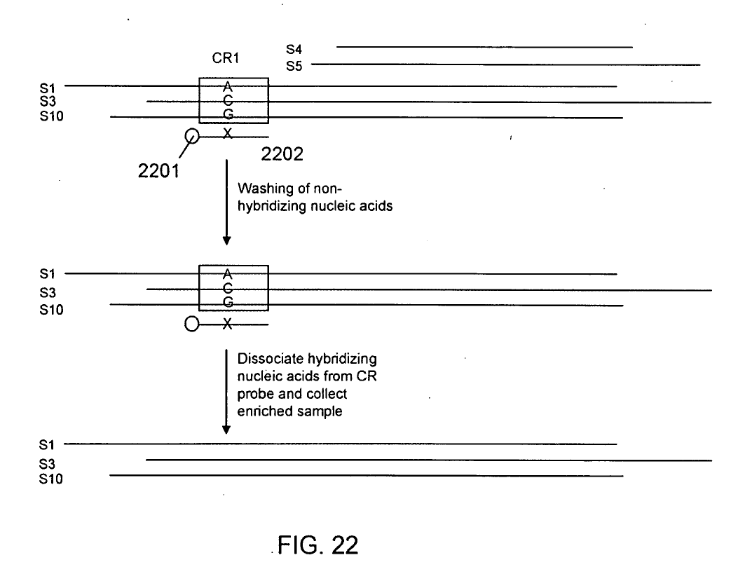

FIG. 23 is an example of Click chemistry reaction.

FIG. 24 is chemical structures of dU incorporated in a nucleic acid polymer chain and dU is modified at 5-position with linker and functional groups which can undergo Click reaction.

FIG. 25 is chemical structures of dU incorporated in a nucleic acid polymer chain and dU is modified at 5-position with linker and functional groups which can undergo Click reaction or coupling chemical reaction with an added linker molecule (L3) carrying dual functional groups for the Click reaction or coupling reaction with the dU unit.

FIG. 26 is a schematic illustration a locked duplex formed due to a covalent link between the two modified residues each from the opposite strand of the duplex.

### DETAILED DESCRIPTION OF THE INVENTION

The present invention relates to devices and methods for high-throughput, long-read, accurate, fast, and low-cost sequencing of DNA. The present invention relates to a next generation long-read sequencing (NG-SS, Next Generation Sanger Sequencing) technology, which utilizes the advantages of time-proven Sanger sequencing and capillary electrophoresis to establish a new platform that will perform microbead-based Sanger sequencing reactions in a massively parallel scale, by separately placing millions of different sequences in a three dimensional (3D) high density capillary module, electrophoretically separate sequencing fragments, rapidly acquiring fluorescence images on the exit plane of the capillary module, and using the rapidly recorded time-resolved images to re-construct sequence information.

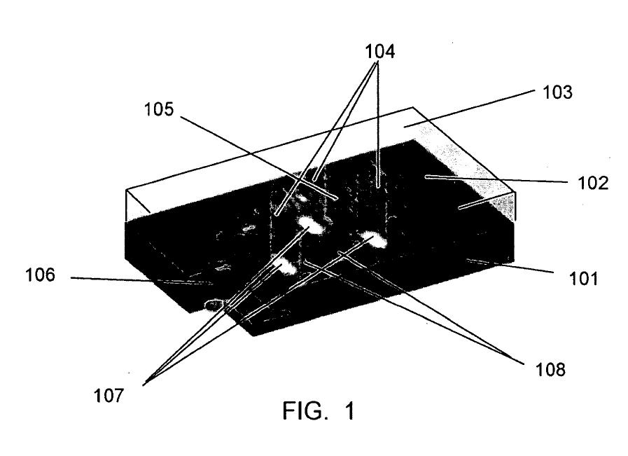

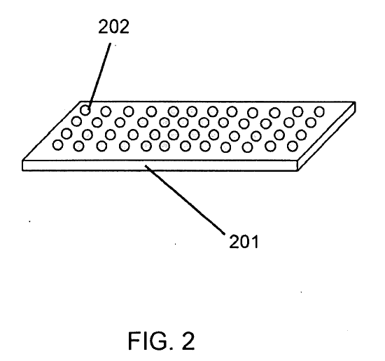

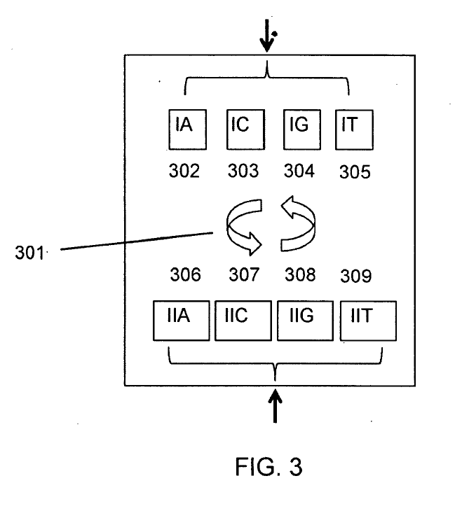

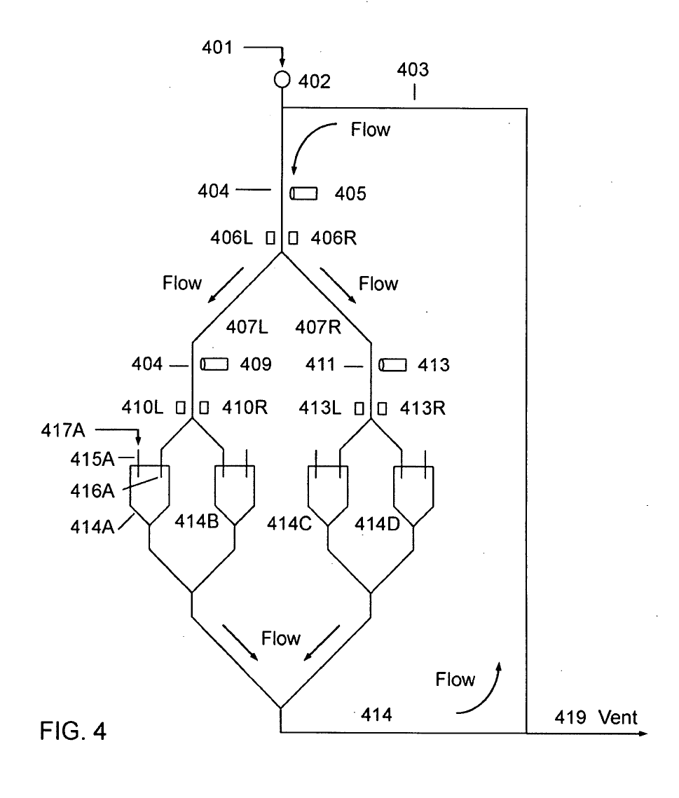

The present invention provides methods and devices for large scale, parallel making of probes and probe beads. In a preferred embodiment of this invention, the method for synthesis of probes is miniaturized in situ synthesis in an array format (FIG. 1 and FIG. 2). Thousands to tens of thousands of probes are synthesized simultaneously in fmol to pmol amounts per each probe and these probes are attached to bead materials to give probe beads. The probes can be but are not limited to DNA, RNA, carbohydrate, peptide, lipid, and small molecules and other chimera of the molecules useful for bioassays. In another embodiment of this invention, a binary sorting synthesis system (FIG. 3 and FIG. 4) and method are provided for rapid parallel synthesis of probes on beads, which are digitally barcoded such that a specific probe be synthesized on each bead according to design. This synthesis method uses beads from nanometers to millimeters in diameter and produces probes in fmol to nmol amounts for each. The present invention provides versatile products for diverse applications of genomics and the related fields of large scale biology.

In this invention, probe synthesis is carried in devices which offer surfaces that can accommodate arrays of molecules. An array contains at least 400 different probes in a square centimeter area, preferably more than 1,000 different molecules in a square centimeter area. Each type of probes is produced in sub-fmols to nanomols concentration, preferably in pmols concentration. FIG. 1 is a drawing of a microfluidic pico-liter array synthesis device (Zhou, X. et al. 2004, Nucleic Acids Res. 32, 5409-5417; herein incorporated by reference). The synthesis of probes may be carried out in parallel in the 200 pL reaction chamber for each probe. At the completion of the synthesis, probes are derivatized with a long linker group bearing a functional group to form a conjugate with the functional group of beads to form probe beads.

In a preferred embodiment of the present invention, a synthesis device such as that shown in FIG. 1. contains about 4,000 reaction chambers. A synthesis device of the type may contain a smaller (i.e., several hundreds) or larger number of reaction chambers (i.e., tens of thousands or more). These reaction chambers may contain a number of beads such as 10 μm Tantagel beads (Polymere GmbH) in reaction chambers. The surface capacity of such a bead allows for more than 10 pmol of molecules to be synthesized, which is about 10,000 fold larger than the capacity of a planar reaction cell of dimension 90×200 μm².

Another method of making probe beads entails adding the units of the sequence (such as nucleotide monomer or amino acids) one by one to the tagged bead and introducing a sorting step between each addition. The sorting step sequesters all the beads which will be subject to the same treatment in the next step, after which the beads can be re-sorted for the next step.

For example, FIG. 3 and FIG. 4 show a preferred method of oligonucleotide nanobead synthesis in which a given molecule can be addressed to a particular tagged bead. Beads can be tagged in a variety of ways including but not limited to fluorescence, radio frequency, molecular tags, molecular sequence tags, optical, magnetic, optomagnetic and combinations thereof. In this method of synthesizing oligo nanobeads, 4 reaction chambers (FIGS. 3. 302 to 305) are filled with tagged, derivatized nanobeads (e.g. OH functionalized Tentagel (10 μm) beads). Each reaction chamber corresponds to one of the four DNA nucleotides A, T, G or C. After a given nucleotide is added to each bead in the reaction chamber, the beads are re-sorted into reaction chambers corresponding to the next nucleotide to be added to the growing sequence. For example, in FIG. 3 eight sequences are listed (see Sequence List). These sequences correspond to eight different molecules to be made. In the first cycle of the 3′-5′ synthesis (the methods of the present invention are not limited by the direction of the synthesis) the nanobeads corresponding to sequences #4 and #8 will start in the chamber IA (FIG. 3. 302) where an adenosine (A) monomer will be added to these beads. In like manner, beads that correspond to sequence #1 will be placed in reaction chamber IC (FIG. 3. 303), beads that correspond to sequences #2, #3, #5 and #6 will be placed in reaction chamber IT (FIG. 3. 305) and beads that correspond to sequence #7 will be placed in reaction chamber IG (FIG. 3. 304). Nucleotides corresponding to the reaction chamber will be added to the beads. In a preferred embodiment of the present invention the nucleotide monomers are conventional monomers which are 5′-DMT protected. After the coupling reaction is complete the beads are then sorted in a process that redistributes the beads in reaction chamber corresponding to the second nucleotide of the desired sequence. For example, in FIG. 3, the beads corresponding to sequence #1 are removed from reaction chamber IC (FIG. 3. 303) and distributed into reaction chamber IIG (FIG. 3. 308) wherein a guanosine nucleotide will be added to the molecule. Beads corresponding to sequence #2 are removed from reaction chamber IT (FIG. 3. 305) and distributed into reaction chamber IIG (FIG. 3. 308) wherein a guanosine nucleotide will be added to the molecule. Beads corresponding to sequence #3 are removed from reaction chamber IT (FIG. 3. 305) and distributed into reaction chamber IIA (FIG. 3. 306) wherein an adenosine nucleotide will be added to the molecule. Beads corresponding to sequence #4 are removed from reaction chamber IA (FIG. 3. 302) and distributed into reaction chamber IIT (FIG. 3. 309) wherein a thymidine nucleotide will be added to the molecule. Beads corresponding to sequence #5 are removed from reaction chamber IT (FIG. 3. 305) and distributed into reaction chamber IIC (FIG. 3. 307) wherein a cytosine nucleotide will be added to the molecule. Beads corresponding to sequence #6 are removed from reaction chamber IT (FIG. 3. 305) and distributed into reaction chamber IIG (FIG. 3. 308) wherein a guanosine nucleotide will be added to the molecule. Beads corresponding to sequence #7 are removed from reaction chamber IG and distributed into reaction chamber IIT (FIG. 3. 309) wherein a thymidine nucleotide will be added to the molecule. Beads corresponding to sequence #8 are removed from reaction chamber IA (FIG. 3. 302) and distributed into reaction chamber IIC (FIG. 3. 307) wherein a cytosine nucleotide will be added to the molecule. The synthesis and sorting cycles are repeated until the desired sequences are synthesized.

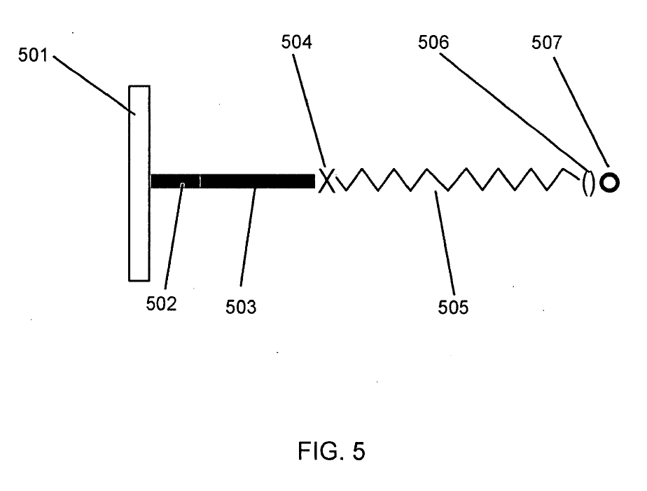

The method of the present invention is not limited by the type of molecules that have been discussed. In preferred embodiments of the present invention DNA, RNA, peptides and carbohydrates or any other molecule that is amendable to in situ synthesis may be synthesized on addressable nanobeads. The methods of synthesis of the present invention are also not limited by the number of reaction chambers that can be utilized in the synthesis of molecular nanobeads. While a single reaction chamber was utilized in the example in FIG. 5, multiple reaction chambers for each monomer species to be added can also be envisioned. Reaction chambers might also be use for more than a single step. Other synthesis protocols including the use of dimer and trimers or longer elements might also be utilized.

The number of different elements to be added will define the minimum number of reaction chambers necessary to have one reaction chamber per element. For example if the synthesis is of a peptide sequence then utilizing the naturally occurring amino acids, 20 different reaction chambers might be necessary for synthesis depending on the length of the sequence.

The synthesis device can have either isolated reaction chambers where the chambers can be physically sealed from one another or the device may have fluid connections between the reaction chambers wherein the beads can flow through a sorting device and be redistributed into other reaction chambers that are in fluid connection with the sorting device.

The addressable nanobeads of the present invention may have a density of 1-1,000,000 molecules per bead. In certain preferred embodiments the nanobead has a single molecule adhered to it.

Nanobeads and other nanoparticles can be modified so that the beads can be sorted by flow cytometry which takes advantage of the rapid (10⁷/min) bead-sorting instruments to generate pools of pre-sorted beads based on a defined set of properties of beads. Such a pool of pre-sorted beads overcomes limitations of the prior art which requires a high level of redundancy in random arrays assembled from a mixture of molecular beads. Pre-sorted beads permit specific beads to be selected for addressable nanoarrays and/or a pool of beads of known sequence contents for specific applications.

The tagged beads may be made into a variety of shapes including but no limited to cylindrical, tubular, spherical, hollowed spherical, elliptic, and disk like. The beads may contain recess structures or areas for protecting active surface moieties from physical contact with other subjects or beads. For example, the beads can be made into dumbbell shape having an active surface area in mid section while both ends of the dumbbell being coated with an inert material. The recessed structures may help avoid bead coagulation and/or damage of active surface moieties. A preferred size of the beads is from 1 nanometer to 1 centimeter in the longest dimension. A more preferred size is from 10 micron to 5 millimeter.

The tagged beads may be made from a variety of materials including but not limited to glass, ceramic, polymer, metal, semiconductor, and combination of more than one material. For example, a bead may contain a paramagnetic core encapsulated with a polymer material. The paramagnetic core facilitates transportation, sorting, and holding of the bead using magnetic force. Another exemplary bead contains a paramagnetic coating, at least on one or more sections of the bead, also to facilitate bead manipulation by magnetic force. Yet another exemplary bead contains a solid core, such as glass, that is encapsulated with a layer of polymer matrix material for increasing synthesis load. The matrix material includes but is not limited to low cross-linked polystyrene, polyethylene-glycol, and various copolymer derivatives (F. Z. Dörwald “Organic Synthesis on Solid Phase: Supports, Linkers, Reactions”, Wiley-VCH, 2002; herein incorporated by reference).

The tag marks on the beads may be produced using a variety of processes that are well-known to those who are skilled in the field of micro-fabrication. One exemplary process is laser marking. Laser marking is well known to those who are skilled in the field of laser processing (J. C. Ion “Laser Processing of Engineering Materials”, Elsevier Butterworth-Heinemann, 2005; herein incorporated by reference). An iron film is coated on a glass fiber by electroplating or by sputtering. The preferred film thickness is between 5 nm to 5 μm. The film coating is well-know to those skilled in the art of thin-film fabrication (R. L. Comstock “Introduction to Magnetism and Magnetic Recording”, John Wiley & Sons, Inc., New York, 1999; herein incorporated by reference). Optical tags in form of coaxial ring barcodes are then laser marked on the fiber surface by ablating the iron film. The fiber is then coated with a protective thin silica film, either by vapor deposition or by sol-gel process (M. A. Aegerter “Sol-Gel Technologies for Glass Producers and Users”, Kluwer Academic Publishers, 2004; herein incorporated by reference). The fiber is cleaved or cut to form a cylindrical bead. The bead is then either derivatized with an appropriate linker moiety or coated with a matrix polymer material. The method shown above is only one exemplary illustration among many variations of bead making processes. For example, the polymer or metal fiber or wire can be used as the core of the bead. The iron film can be replaced with a paramagnetic iron oxide or nickel phosphorus film. A dark color metal oxide film can be deposited on top of magnetic film to produce a high-contrast barcode by laser marking. The fiber can be cleaved or cut after linker derivatization or matrix polymer coating. The coating of a fiber with a matrix polymer can be done in a similar way as that of putting a cladding layer on glass fiber for making optical fibers.

FIG. 4 is a schematic diagram of an exemplary binary sorting synthesis system. The system uses magnetic beads that contain optical barcodes. Before the start of a synthesis, a group of beads, each having a known barcode, is selected. Each bead is assigned with a sequence to be synthesized. At the beginning of a synthesis reaction beads-containing solution 401 is sent into the system through an entrance port 402. When a bead passes through detection port 404 its barcode is read by optical sensor 405. Depending on the barcode and its designated sequence, electro-magnetic field generator 406L or 406R is activated to cause the bead flowing either into flow channel 407L or into channel 407R so as to complete level one sorting. Level two sorting is done in a similar fashion and through detection ports 408 and 409, optical sensors 409 and 413, and electro-magnetic field generators 410L, 410R, 413L, and 413R. The bead is eventually steered into a designated reaction chamber (414A, 414B, 414C, or 414D) in which a specific sequence residue is to be added to the molecular moiety on the bead. While not shown in the figure, a mechanism is available in each reaction chamber to hold the bead inside the chamber. Exemplary holding mechanisms include but are not limited to mechanical stoppers and magnetic fields. When all the beads have been sorted and placed into designated reaction chambers reaction reagents (e.g. 417A) are sent into the reaction chambers (414A, 414B, 414C, and 414D) through reagent deliver lines (e.g. 415A) to carry out a synthesis cycle. Reacted reagents are discharged through venting line 419. Upon the completion of the synthesis cycle beads are released from all reaction chambers and are pushed into a circulation line 418. With venting line 419 closed (venting valve is not shown in the figure), the beads are then returned back to level one sorting through returning line 403. The next sorting and synthesis cycle can then begin. The synthesis cycles are repeated until all designated sequences are synthesized. The present invention may be used with any known solid-phase and combinatorial synthesis process (U.S. Pat. No. 7,190,522 and references; herein incorporated by reference).

The flow channels shown in FIG. 4 can be made of glass, plastic, silicon, or any appropriate materials. The size of the channels may vary from sub-micrometer to millimeters in diameter depending on applications. For synthesis on small beads, the preferred flow channel diameter is between about 1 to about 200 micrometers. The channels can be fabricated using etching process on glass or silicon wafers. Reaction chambers (414A, 414B, 414C, and 414D) can be formed on the same wafers. For synthesis on larger beads, such as matrix polymer encapsulated beads, the preferred flow channel diameter is between about 100 micrometers to about 1 millimeter. Conventional tubing, made of glass, fluoropolymers, or other types of chemical resistant materials can be used. Reaction chambers (414A, 414B, 414C, and 414D) can be made of chemical resistant polymers such as fluoropolymers and polyphenylene sulfides, glass, or stainless steels.

The binary sorting synthesis system shown in FIG. 3 and FIG. 4 is only one exemplary illustration among many variations. For example, a buffer chamber can be placed between returning line 403 and detection port 404 to better regulate bead flow. A movable frit filter disc can be placed at the bottom of each reaction chamber (414A, 414B, 414C, or 414D) and a reagent delivery line can be placed below the filter while substrate beads lay above the filter. With this arrangement, a chamber reactor operates in a float-bed manner and good mass transfer can be achieved during synthesis reactions. Additional sorting levels can be added to meet the requirement of additional distinct residues such as in case of peptide synthesis. In a preferred mode, optical sensors 405, 409, and 413 are photodiodes. In another preferred mode optical sensors 405, 409, and 413 are CCDs (charge-coupled devices). In certain operational modes, for example when bead flow rate inside sorting channels is stable or predictable or when the time interval between two adjacent beads are sufficiently long so that the second bead enters into detection port 404 after the first bead has entered its designated reaction chamber, only one optical sensor 405 may be needed. While not shown in the figure, illumination lights may be used in conjunction with optical sensors. The optical sensors (405, 409, and 413), magnetic field generators (406L, 406R, 410L, 410R, 413L, and 413R) and fluid controls valves (not shown in FIG. 4) can be in communication with one or more computers and their signal collection and/or movement actuations are controlled by the computer. Other bead encoding and decoding methods can be used. For example, magnetic encoding and decoding methods can be used. In this case, a magnetic recording head is placed on the side wall of a flow channel. Binary codes can be written or read to or from a paramagnetic film coated bead in the same way as that of digital recording using one or more magnetic taps or discs.

Beads can be manipulated by forces or effects other than or in addition to magnetic force. For example, using piezoelectric devices, mechanical deformation can be created inside fluid channels so as to steer the flow direction of beads. Heat, produced by laser or resistive elements, can be applied to flow channel wells and to cause flow disturbance so as to affect the flow direction of beads. A computer controlled 1D or 2D transportation arm in conjunction with a code reading device can be used to deliver tagged beads to designated reaction chambers instead of using the binary tree sorting mechanism shown in FIG. 4. The present invention significantly increases the speed of synthesis by reducing the overall operation steps and using the advanced microparticle sorting technologies. Bead selection at each reaction cycle for synthesis is processed at a speed hundreds to million per second.

In an embodiment of the present invention, after the completion of synthesis of all designated sequences, the barcoded beads can be used for performing assays on the bead surfaces or can be used for producing materials by cleaving the synthesis products from the beads. The matrix polymer encapsulated beads are particularly suitable for producing off-bead synthesis products. Individual sequence products can be produced by placing the barcoded beads into cleavage reaction wells, which can be in 96-well format, 384-well format, 1536-well format, or certain custom-made format, and perform cleavage reaction in parallel. The placement of the barcoded beads can be done using a computer controlled transportation arm in conjunction with a code reading device. A mixture product can be obtained by placing all or a selected number of beads in a cleavage reaction well and performing a cleavage reaction. These syntheses produce fmol to nmol per sequence materials, preferably, pmol to a few nmol of materials with a few thousandth or less solvent consumption as conventional one-by-one oligo synthesis such as that process used by Illumina (www.illumina.com) to produce oligo beads for bead microarrays.

In this invention, beads for loading probes have various properties. The sizes of beads preferably are in the range of a few nanometers to millimeters, and beads of one micron or so are preferably used in the array synthesis device. Beads of a few micron to millimeter diameter are preferably used in the binary sorting synthesis system. The shape of beads or nano- and micro-particles can be spherical, elongated, cylindrical, and other irregular shapes. At the bead surface there can be coating layers of porous and/or non-porous particles to give desirable surface synthesis and/or attachment properties. The surface can be functionalized as carriers of assay probes. Different kinds of beads are applicable for making probe beads, including but not limited to silica beads (e.g. those from Bands Laboratories, Inc.), magnetic beads (e.g. those from Invitrogen/Dynal beads), polymeric beads (e.g. those from Rapp Polymere). In the present invention four types of beads and the corresponding chemistry are preferred: gold or gold coated spheres (10-100-nanometer, thiol group), avidin/streptavidin coated magnetic beads (<10 biotin group), TentaGel beads (Rapp Polymere GmbH, Germany, 1-100 μm, 3, 10, 30 μm, NH2 or OH conjugation chemistry), Sephadex beads (20-50, 40-120 μm, carboxyl, NH2 conjugation chemistry). Beads may contain tags/markers for detection and identification, such as fluorescence molecules (Fluoresbrite polystyrene beads (Polysciences), luminescence molecules, chromophore molecules, magneto electronic group/print, quantum dots, biotin, etc. In this invention, beads used in the microfluidic array reactor shown in FIG. 1 are made of stable materials including, CPG (controlled pore glasses), cross-linked polystyrene, and various resins that are commonly used for solid-phase synthesis and analysis.

The present invention relates to solid surface (FIG. 5, 501) synthesis of probe molecules which may contain surface linker and spacer groups such as alkyl, polyethylene glycosyl chains. The linker group (FIG. 5, 501) is an anchor point for attachment on surface and spacer (FIG. 5, 502) provides the accessibility and structural flexibility for probes (FIG. 5, 505) to interact with target molecules. Probe molecules may contain tags (FIG. 5, 507) through conjugation (FIG. 5, 506), such as those fluorescence molecules, chromophore molecules for detection, biotin which can link to a detection molecule, or a bead moiety (FIG. 5, 507). Probes may be cleaved at a specific cleavage point (FIG. 5, 504). In one embodiment of the present invention the cleavage point (504) is dU (cleavable using USER kit from New England Lab (NEB)), conjugation site (506) is a biotin and streptavidin linkage and this is linked to a nanobead (507) which is linked to streptavidin.

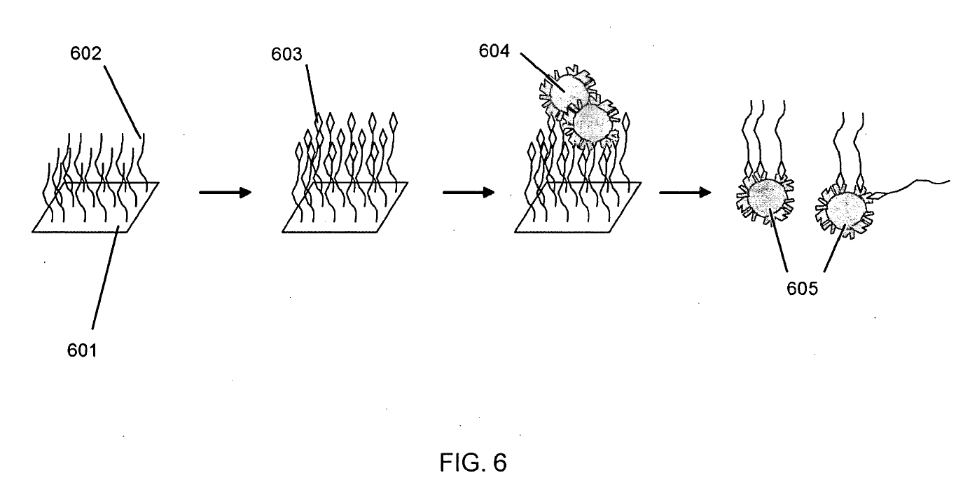

The present invention also relates to the conjugation reaction for joining two kinds of molecules, or a molecule with beads, or beads with surface. Specifically, oligos can be attached to a surface or beads and beads in solution attached to the surface oligos. Bead surface reactions are traditionally carried out using molecules in solution and functionalized to react with a bead surface. A number of chemical methods for conjugation are suitable choices for these purposes (Kozlov, I. A. et al., 2004, Biopolymers 73, 621-630; Soellner, M. B. et al., 2003, J. Am. Chem. Soc., 125, 11790-11791; Houseman, B. T. et al., 2002, Nat. Biotech. 20, 270-274; Farooqui, F. and Reddy, P. M., 2003, US 2003/0092901; Wang, Q. et al., 2003, J. Am. Chem. Soc., 125, 3192-3193; Clarke, W. et al., 2000, J. Chrom. A, 888, 13-22; Raddatz, S. et al., 2002, Nucleic Acids Res. 30, 4793-4802; Konecsni, T, and Kilar, F., 2004, J. Chrom. A, 1051, 135-139; herein all incorporated by reference). In one embodiment of the present invention, an array of more than 100 oligonucleotides is synthesized on surface and the terminal group, preferably the 5′terminal group, is an alkylbiotin. A solution of streptavidin coated magnetic beads (e. g. Dynabeads® M270 Streptavidin) is added to the surface. Biotin and streptavidin are high affinity binding pairs (Kd>10¹³ M) and the solution and surface contact results in the beads binding to oligos on surface. In case when the dimension of a reaction site of oligo synthesis is much greater that the size of the bead, one bead will be surrounded by the same oligos in the reaction site (FIG. 6). In certain embodiments the biotinylated oligos that are conjugated to strepavidin beads are the same sequence to give one-bead-one-type of oligo probe beads.

The present invention also relates to the conjugation reaction for joining two molecules, or a molecule with beads, or beads with surface. Specifically, oligos can be attached to a surface or beads and beads in solution attached to the surface oligos. The conjugation reactions can occur between a pair of reactants (the first and the second functional groups from the pair of reactants) and also between multiple pairs of reactants (the third and the fourth functional groups of the second pair of reactants). The functional groups include reactive groups and high affinity binding groups, such as alkynyl, alkylazide, amino, hydroxyl, thiol, aldehyde, phosphoinothioester, maleimidyl, succinimidyl, isocynate, ester, hydrazine, strepavadin, avidin, neuavidin and biotin binding proteins. In a conjugation reaction, wherein the first functional group is biotin and the second functional group is strepavadin, avidin, neuavidin or other biotin binding proteins; in another conjugation reaction, wherein the first functional group is alkynyl and the second functional group is azide; in another conjugation reaction, wherein the first functional group is amino and the second functional group is ester, succninimidyl, or isocynate; in another conjugation reaction, wherein the first functional group is thiol and the second functional group is phosphoinothioester, maleimidyl; in another conjugation reaction, wherein the first functional group is hydroxyl, and the second functional group is ester, succinyl, succninimidyl, or isocynate; in another conjugation reaction, wherein the first functional group is aldehyde, and the second functional group is amine, or hydrazine. For the pair of functional groups, e.g. the first and the second functional groups are interchangeable as to the attached functional group. There is no limit to the functional groups contained in a molecule and thus one or more conjugation reactions are possible between a pair of molecules and/or substances.

There are many methods for conjugation of two molecular entities, and the basic requirements for practical usefulness are: (a) the resultant conjugate is suitable for further applications, (b) conjugation reaction sites should be easy to prepare, (c) the reaction should cause minimal side and/or nonspecific reactions, and (d) reaction time should be reasonably short. In the present invention four types of beads and the corresponding chemistry are preferred: gold (nanometer, thiol group), streptavidin coated magnetic beads (<10 μm, biotin group), TentaGel beads (Rapp Polymere GmbH, Germany, 10 μm, NH2 or OH conjugation chemistry), Sephadex beads (˜25 μm, used by 454 Sequencing technology, NH2 conjugation chemistry). Streptavidin coated magnetic beads are widely used for separation of different sequences through biotin-tag selection; the method is useful for purification, enrichment, separation, and other applications. Biotin functionalization of oligos may be accomplished by using standard phosphoramidite chemistry using a biotin-modifier agent. (Glen Research). This is a phosphoramidite agent and thus it can be coupled to the 5′-OH of an oligo after the full-length sequence is synthesized. Certain biotinylation agents permit coupling of a fluorescent dye after the biotinylation agent is coupled to the surface oligos. Such a fluorescent label can be used to validate the incorporation of the biotin moiety. Fluorescein molecules can be as a monitoring tool for synthesis and therefore can provide guidance for optimizing the biotinylation reaction.

The present invention includes a method of making addressable probe nanobeads mixture wherein each nanobead is attached to a single type probe molecule comprising: a) synthesizing an array of probe molecules on a surface wherein the molecule has a first terminus and a second terminus and wherein the first terminus is attached to a spacer that is attached to the surface and the second terminus can be coupled to a first functional group; b) conjugating a functional group to the second terminus; c) coupling tagged nanobeads that have been derivatized with a second functional group to functional group on the second terminus of the probe molecule; d) removing the uncoupled tagged nanobeads from the surface; e) capping the functional group of the uncoupled probe molecules; f) cleaving the tagged probe nanobeads from the array to form a mixture of addressable probe nanobeads mixture wherein each nanobead is attached to a single type probe molecule. The arrays of the present invention may comprises more than 1000 different probe molecules. In preferred embodiments the spacer has from 6-30 chemical bondsand is coupled to a cleavage site such that the addressable probe nanobead can be cleaved from the surface. Functional groups can be but are not limited to biotin, hydrazine, alkynyl, alkylazide, amino, hydroxyl, thiol, aldehyde, phosphoinothioester, maleimidyl, succinyl, succinimidyl, isocynate, ester, strepavidin, avidin, neuavidin and biotin binding proteins. Nanobeads can be treated with protein and surface blocking solution (such as 0.5% BSA in PBS buffer) to prevent nonspecific binding before conjugation with the probe. Blocking proteins or nonionic surfactants can be used to reduce the background non-specific interactions. A stringency wash step can be carried out using diluted reaction solution or a solution with increasing dissociation power. This further removes the beads retained on surface due to nonspecific interactions and increases the ratio of correctly conjugated beads to non-specifically bound beads. The various reaction conditions, (e.g. buffer, solvent, temperature, pH and time) may have significant effects on the conjugation reaction. In preferred methods of the present invention the probe is preferably DNA oligonucleotides of 10-200 residues, and/or RNA oligos of 10-200 residues, and/or DNA and RNA chimer (mixes composition of DNA and RNA) 10-200 residues.

Functionalization can be accomplished by chemical conjugation. One widely used method is to generate an amino group such as by incorporation of an amino modifier or a 5-(3-aminoallyl)-dU into the oligo sequence or coupling an amino-linker moiety (FIG. 5) to the 5′-OH group using a phosphoramidite (Glen Research). The 5′-terminal amino group of the oligos can react with an activated ester, such as an NHS ester coated on the surface of beads to form an amide bond. The conjugate oligo-bead is stable in most chemical and bioassay conditions. The functionalization does not necessarily require the 5′-terminal amino group of oligos; else where in the oligo chain, suitable modifications as discussed for conjugation chemistry in the prescribed invention can be incorporated. Intermolecular conjugation linkage can be formed between the modification groups.

In an another embodiment of the present invention, functionalization can be accomplished by an adsorption method. The oligo can be modified, using 5′-thiol modifier (Glen Research), to a thiol group such that the oligo contains a SH moiety. SH has high affinity to gold surfaces. Gold spheres containing immobilized oligos have been successfully applied in assays of DNAs and in nanostructure constructions. Preferred functionalization chemistries are compatible with oligo synthesis/deprotection chemistry and these functional groups are commonly used as modifiers for oligo immobilization onto solid surfaces. The surface linkage chemistry suitable for synthesis and also removal of bead-tagged oligonucleotides from surfaces may be optimized to improve the efficiency of the generation of probe bead mixes.

The present invention also relates to methods for the conjugation reaction of a surface and beads which are in solution. In one embodiment of the present invention, the bead surface is derivatized with oligoethylene glycosyl amino spacer group. The total chain length of the spacer measured by number of bonds is greater than 6, and preferable is greater than 18 and more preferably greater than 30. The beads in coupling reaction solution (DIC/DMAP (1,3-diisopropylcarbodiimide/dimethylaminopyridine) in DMF/CH₂Cl₂) contain surface succinyl which can react with the surface linker. After the reaction, the beads are retained on the surface when the surface is washed multiple times. In comparison, the beads which do not have the surface succinyl group are washed away since there is no covalent bond formed between the beads and the surface.

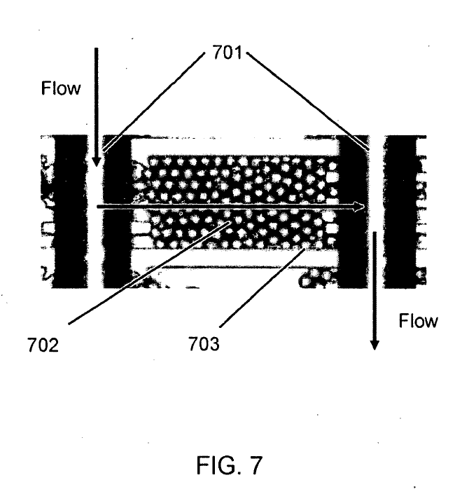

In an embodiment of the present invention, the surface to which the beads are attached is comprised of three dimensional reaction chambers as depicted in FIG. 1 and FIG. 7. The beads are adhered to the reaction chambers through conjugation reaction with the chamber surface so that they are not stripped from the surface as fluid flows through the channel (FIG. 7, 701) to chambers during multiple steps of chemical synthesis reactions (FIG. 7, 702). The beads are also confined to the chamber by the separation walls on both sides of the chamber aligned orthogonal to the flow channel (FIG. 7, 703). The methods of the present invention also provides for optimization of bead surface functionalization, thereby providing high quality synthesis results. The reaction chamber dimensions are 10 to 500 microns, which are larger than the bead sizes (10 nm to a few hundred μm) such that a large number of beads can be immobilized in each reaction chamber such that sufficiently large numbers of molecules (e.g. fmol to nmol, preferably pmol to nmol) are synthesized per array synthesis.

In one preferred embodiment of the present invention, FIG. 1 depicts a three dimensional microfluidic pico-array device comprising three dimensional reaction chambers each having a surface area of approximately 90×180 mm² and a height of 16-30 μm. The array illustrated in FIG. 1, contains 3,968 reaction chambers that can accommodate 3,968 independent synthesis reactions. Based on the above referenced dimensions for the reaction chamber and the use of 1 μm beads filling 20% of the reaction chamber capacity each reaction site can accommodate about 8,100 or more beads.). At this level, one chip synthesis can generate beads for several hundreds to at least one thousand assays at pmol level.

It is realized that on a glass plate synthesis device (FIG. 2), probe synthesis is not restricted to a chamber for beads to be attached to the surface (FIG. 6) or probes cleaved to be used as a mixture of molecules or probe beads after attaching the cleaved molecules to beads added to the probe solution.

Depending on the size of the beads and the application an array having reaction chambers of this size can accommodate millions of beads. The microfluidic device can be scaled to increase or decrease the size of the reaction chambers according to application requirements. In a preferred embodiment the synthesis of molecules on the attached beads is performed using projection light which is digitally controlled and reaction reagent (PGR) forms under light irradiation (Gao X., et al., U.S. Pat. No. 6,426,184, Gao X., et al., U.S. Pat. No. 7,235,670; herein incorporated by reference). The light triggers chemical reaction on beads in the reaction chambers which are irradiated. Biopolymers may be synthesized by repeating the steps of light irradiation, deprotection, and coupling reactions. Beads conjugated to an array chip synthesis device is shown in FIG. 7 where 10 μm TentaGel beads were loaded on to a microfluidic chip in a dispersed mode, and the beads were reacted with a succinyl group on the chip surface thereby immobilizing the beads on the chip surface. The optical unit power for delivering suitable light strength and fluidic delivery for reactions occurring in reaction chambers filled with nanobeads need to be tailored to array synthesis. In general, irradiation power in the range of tens of mW to hundreds of mW at the position of the synthesis surface is desirable; sufficient amount of photogenerated reagents formed for the deprotection reaction.

In the present invention, one of the applications of the methods of making molecules on beads contained within an array is to increase the yield of the molecules. Present arrays can only make about 1 fmol of oligomer per reaction chamber. With the bead synthesis methods of the present invention about 1 pmol to about 20 pmols per reaction chamber can be produced. Furthermore with an array structure about 4,000 to about 100,000 different DNA oligos of these quantities can be made per array. The increased capacity allows researchers to utilize subsets of probe bead oligos to focus sequencing results on the areas of particular interest.

In the present invention, one of the applications of the methods of making molecules on beads contained within an array is to increase the yield of the molecules. In an embodiment of the present intention, one reaction site uses pseudo-codon (Gao, X. et al., WO2008/003100.) (pseudo-codon is a symbol, such as Z, which can represents more than one monomer building blocks in a synthesis, e.g. Z=A and G and this information is used for synthesis by a synthesizer. Adding a mixture of monomers to the synthesis results in formation of two or more compounds, depending on the number of monomers that the pseudo-codon includes. The use of multiple pseudo-codons results in formation of combinatorial libraries. For instance, for a oligomer synthesis, if the first pseudo-codon represents 3 monomers, and the second pseudo-codon represents 3 monomers, the synthesis of this oligomer results in a library of 9 different compounds). Thus, multiple different molecules can be made on a single reaction site. This form of synthesis is greatly benefit from the methods and devices of the present invention. The amount of each molecules in the library synthesis is greater than what obtained from a conventional synthesis.

In another embodiment, the present invention provides methods and devices for attaching beads to molecules that have been synthesized on a surface (FIG. 7). The molecules to which the beads may be attached include but are not limited to DNA, RNA, PNA, lipids, peptides, proteins, and carbohydrates. The bead may be attached by functionalizing a position or multiple positions on the terminus or within the molecule to generate a reactive site capable of affinity binding or covalent bonding with a separate molecule or a bead. In the present invention the preferred method is to functionalize the terminus such as the 5′ end of an oligo) however functionalization may be selected at any position(s) on the molecule to be synthesized. A benefit of 5′-functionalization for oligomers is that synthetic failure sequences are capped after the last step of coupling and thus are no longer available for functionalization. The quality of the collected 5′-functionalized sequences is thus improved.

After cleavage the bead probes can be collected and formulated into a mix. In the case where oligo molecules are to be cleaved from the synthesis surface the oligos may contain several functional sites (FIG. 5. Each oligo contains at least one cleavage site [designated X, FIG. 5], a 5′-functionalization site [designated ( ) FIG. 5] and a bead conjugation site [designated (O), FIG. 5]. But the functional groups are not limited to the terminal positions and are synthesized at different positions in the probe molecule. The cleavage site for releasing surface molecules into solution is specifically designed so that desired molecules can be obtained for further applications. But it is also possible to use a general base or acid condition to cause the detachment of the probe molecules from surface. It is also possible to use an enzymatic condition to cause detachment of the probe molecules from surface. The probe bead cleavage site should be stable under synthesis conditions. The probe bead cleavage site should be able to be cleaved after the oligos are synthesized. Normally, the cleavage of oligonucleotides synthesized on a solid support, such as controlled porous glass (CPG), is accomplished by liquid ammonia hydrolysis of an ester bond. However, in array oligo synthesis, the synthesized oligos should remain on surface for assay applications, and thus it is not practical to use the same surface linkage chemistry as used in CPG oligo synthesis. U.S. Pat. No. 7,211,654, (Gao X., et al., herein incorporated by reference) describes a method for cleaving oliogos from synthesis surfaces; incorporated by reference. The cleaved oligos have 3′-OH groups and the OligoMix™ thus generated has been used in a variety of applications, such as primers, cloning inserts for mutagenesis and siRNA sequence libraries. The rU chemical modification can be used in either nuclease enzymatic reactions or base hydrolysis conditions for cleavage. These reactions are compatible with conjugation bonds and complexes such as biotin-streptavidin or covalent amide linkages. In a preferred embodiment of the present invention, the probe bead oligos contain an rU linkage. The rU monomer phosphoramidite can be incorporated in the oligo synthesis on surface. The cleavage reaction conditions can be optimized based on the specific type of the probe bead mixes.

In general, reactions are more efficient if the surface face oligos are more “solution-like”. Therefore, in preferred embodiments of the present invention linker and/or spacers are utilized to achieve more efficient reactions. In one embodiment of the present invention, the linker unit is a propylamine. The spacer unit is flexible due to the chain length. Hexaethylene glycol may be used as building blocks for the spacer. Optimization of spacer length is achieved by comparison of sequence sets containing different spacer lengths at different reaction sites on the same chip. The detection of fluorescence signal strength gives information on spacers which produce efficient synthesis (they have stronger fluorescence signals).

In a process of preparing a bead probe mix which includes oligo synthesis (FIGS. 6, 901 and 902), oligo functionalization (FIG. 6, 903), oligo bead conjugation (FIG. 6, 904) and bead probe removal (FIG. 6, 905). The probe bead mix which may contain a large number of different sequences may be used for various applications including target-specific sequencing and target specific amplification. The oligos can be capture-probes (i.e. to hybridize and subsequently the duplexes are removed from the sample or primer-probes (i.e. as PCR or other amplification method primers) for amplification of a specific genomic region, and for amplification of genes such as cancer-related genes.

The probe beads of the present invention may also be made by array synthesis (parallel and in large number of different sequences) of molecules as depicted in FIGS. 6 (901 and 902). which are then cleaved from the synthesis surface and subsequently mixed and attach to beads through conjugation.

Probe beads created can be utilized in bead, preferably nanobead, tagging, labeling and sorting, nanoarray assembling and other applications where beads are used individually or as a set of mixtures. Bead tracking and sorting methods of the present invention provide flexible and diverse applications of nanobeads. Addressable nanobead arrays may be created by using sorted nanobeads or by bead-tagging and tag-detection. Methods of nanobead tagging include oligonucleotide coding of each bead, sequencing decoding and multi-fluorescent tags or internally optically coded beads used in a combinatorial fashion (this now can be handled as subsets by flow cytometry). These methods of tagging the nanobeads permit easily assemblage of custom, addressable nanoarrays according to user's designs. These nanoarrays generated by the method of the present invention provide much greater diversity than microarrays presently available.

The nanobead arrays or a mixture of probe beads of the present invention may contain mixed molecular beads. For instance, profiling or detecting a broad line of cellular proteins will provide key information for many biomedical tests. This is presently not possible since there are no tools which are capable of simultaneously detection of different proteins. However, the nanoarrays or a mixture of probe beads of the present invention provide an array with different molecular probes thereby enabling a method for simultaneous detection of multiple different types of molecules in a sample, such as nucleic acids and proteins. For instance, comprehensive detection of proteins may be achieved by a nanoarray of molecular probes consisting of DNA and RNA for detection of nucleic acid binding proteins, peptides as substrates for their cognate proteins and enzymes (e.g. kinases and proteases).

The methods and compositions of the present invention provide high quality synthesis of oligonucleotides on chip and also provide methods of monitoring the synthesis procedures. The monitoring provides for control and continuous improvement in the quality of oligos. Several methods are effective in evaluate the quality of synthesis. Direct fluorescence residue coupling in oligos of different lengths These reactions can be performed under low fluorescence concentrations to avoid saturation of the dye molecules on surface Hybridization using well-characterized control sequences to obtain perfect match (PM) and mismatch (MM) ratios. Cleavage and sequencing of long oligos made on surface. Finally, capillary electrophoresis analysis of the single sequence synthesized on an array.

While the preferred methods of making the nanobead arrays and probe beads mixes of the present invention use Photogenerated Reagent (PGR) chemistry and microfluidic array (μParaflo®) technology, methods and devices of the present invention are applicable to a variety of current DNA microarrays, including the microfluidic pico-array platform (4,000-30,000 features on a single array), other low to high density microarrays, (40,000>1 million features on a single array), Agilent arrays (40,000-200,000 features), Affymetrix/Nimblegen arrays (250,000>1 million features), Febit arrays of Nimblegen-type technology (8,000-40,000), or BioDiscovery's glass plate arrays (>40,000 features) synthesized using PGA chemistry. All of these current technologies can be adapted to suitable bead-conjugation (with modification chemistry development) to generate comprehensive probe bead mix products. Beads utilized in the methods and devices of the present invention include those of different sizes (submicron to 30 μm) and made from different materials, including but not limited to gold, polystyrene, sephadex, and grafted polyethylene glycol and polystyrene. The bead-loading, surface interactions, specific affinity binding or covalent bonding may be systematically optimized to maximize the conjugation of beads to oligos and minimize side reactions. The probe beads obtained from the methods discussed are in smaller quantities in the amount of about 0.1 fmol.

In preferred embodiments of the present invention the beads in the chip are present in the form of a monodispersion. To achieve a monodispersion several factors should be considered. Solvents (e.g. dipole, density, viscosity, temperature, etc.), solvent pH, and bead handling (concentration, method of mixing, open or closed surface, etc.) have effects on the creation of a uniform bead distribution on surface.

In some embodiments of the present invention it is desirable to maximize the number of sequences made per unit area. While an increased sequence density is not necessarily a positive factor for hybridization microarrays, for probe bead oligos, it is useful for increasing the copies of the oligos synthesized so that more sequences can be recovered from a given area. Dentrimer phosphoramidites such as trebler (Glen Research, Trebler Phosphoramidte) is selected as one of such examples, which couples with a surface OH group and, after deprotection, generate three OH groups, which can subsequently couple with three phosphoramidite molecules in next reaction step. Measurement of the oligo yield generated (determined by fluorescein coupling to the 5′-terminus of the sequence) as a function of the generations of trebler coupling gives 3×3, 9 times of the original OH numbers. The dentrimer method is limited by the steps the dentrimer can add before surface molecules saturate the surface or before surface becomes to be too crowded.

In an embodiment of the present invention, the probes and probe beads are used to generate oligo library in the form of droplet. A solution is made at a concentration of about nM (nanomolar) so that each droplet contains one types of probe or probe bead. Using the instrument from RainDance (http://www.raindancetechnologies.com/applications/next-generation-sequencing-technology.asp), the droplet of the sample and the droplet of the specific oligonucleotides are mixed and the probes selected for enrich specific genetic regions are PCR primers to allow sequence-specific sequencing and other genetic analysis.

### Bead Based Sanger Sample Preparation

An essential and common approach in all the next generation cyclic sequencing methods is the use of in vitro single DNA molecule amplification, either by emulsion PCR (emPCR) in a tube or bridge amplification on a glass surface to obtain enough molecules for fluorescence detection. In the present invention the use of emPCR is extended to bead-based Sanger amplification reactions. While the conventional low throughput Sanger sequencing method relies on cloning and/or PCR and one Sanger reaction per tube (or per micotiter well), the methods of the present invention utilizes tens to hundred of thousands or more of individual reactions in a single PCR tube. This significantly shortens sample preparation time, and produces a hundred thousand or more fold reduction in reagent consumption, thereby reducing costs on robotic instrument and supplies. In one embodiment of the methods of the present invention a two step emPCR is employed to ensure the generation of a sufficient number of target molecules for detection since sequencing amplification reactions using di-deoxy NTPs (ddNTPs) usually has a amplification factor less than 100. In preferred embodiments of the present invention a one step emPCR method is employed.

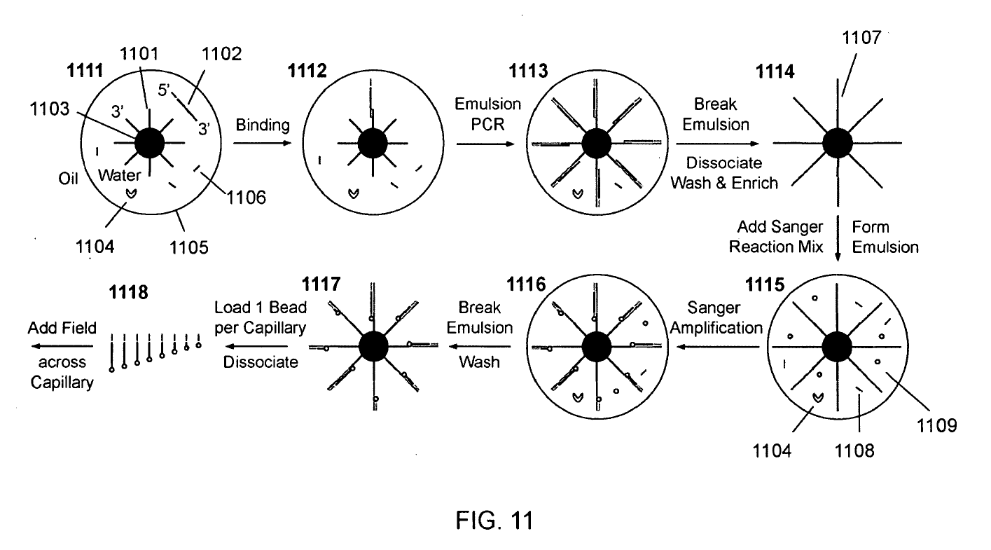

FIG. 11 shows the process flow of one embodiment of sample preparation method of the present invention. Steps in this embodiment include but are not limited to emulsion PCR amplifications, for both template amplification (steps 1111 to 1114) and Sanger amplification (steps 1115 to 1118). For genomic DNA sequencing, genomic DNA is first fragmented by shearing and then common adapters are attached to the fragments by ligation forming PCR templates (not shown in the figure). The templates are added to a PCR mix containing polymerase 1104, dNTPs 1104, and reverse primers 1106. The concentration of the templates is optimized such that when emulsion is formed on average each bead-containing-water-phase droplet contains one template molecule 1102. In one embodiment, each bead 1103 is covalently attached with multiple copies of forward primers 1101. In another embodiment, each bead 1103 is covalently attached with multiple copies of forward and reverse primers. In yet another embodiment each bead 1103 is covalently attached with multiple copies of PCR primers as well as a capture sequence that is designed to capture specific or all PCR products by hybridization. In a preferred embodiment, the attached PCR primers have 3′-OH groups and have their 5′ ends attached to the bead surface. The primer/capture containing beads 1103 are added into the PCR mix solution. An oil solution is prepared by adding surfactants (e.g. 1% Sun Soft No. 818SK) and co-surfactants (e.g. polyglycerol esters of inter-esterified ricinoleic acid) into mineral oil. In one embodiment a water-in-oil emulsion solution is formed by adding the aqueous PCR mix into the oil solution (>70% oil) under stirring using a magnetic bar. In another embodiment a water-in-oil emulsion solution is formed by using mechanical shaking and/or subjecting to stir conditions such as using steel beads. Various methods of emulsion PCR are well described in a number of publications, such as the ones by Margulies 2005 and Kojima 2005, which are included as reference. Schematic drawing of step 1111 of FIG. 11 shows the contents in a most preferred droplet form which contains one template DNA molecule 1102 and one bead 1103.

Emulsion PCR amplification (steps 11122 and 1113 of FIG. 11) is designed for clonal amplification of single DNA molecules to produce beads with each containing amplicons of a single sequence. The amplification is performed in PCR tubes using a regular PCR thermocycler. Each tube may contain about 10,000 to 1,000,000 beads with bead size ranging from 1 μm to 100 μm in solution ranging from 20 μL to 100 μL. In another embodiment the PCR reaction is performed in 96, or 384 well titer plates. The design of a PCR thermocycling program will include consideration of maximizing the length and yield of full length sequences of the surface-bond first stand DNA 1107 that will be used as the template for producing Sanger sequencing fragments. This may be done by adding 10 to 15 cycles (e.g. 30 seconds at 94° C., 360 seconds at 58° C.) of hybridization-extension (to populate full length amplicons) after 40 cycles (e.g. 30 seconds at 94° C., 60 seconds at 58° C., 90 seconds at 68° C.) of regular amplifications. After the completion of PCR reaction isopropanol or another solvent such as ethanol can be added to break the emulsion. The beads will then be washed by isopropanol or another solvent such as ethanol followed by an annealing buffer. The second strand DNA on the beads will be removed by incubating the beads in a basic solution. In one embodiment about 30% of the resulted beads will contain amplicons and the rest of the beads will have no sequence attached. In another embodiment about 50% of the resulted beads will contain amplicons. In another embodiment about 70% of the resulted beads will contain amplicons. A yield of 30% although sounds low but is actually reasonable and acceptable since we want to keep the initial template concentration sufficiently low to avoid the inclusion of more than one DNA template molecule per droplet. In a preferred embodiment a process is included to enrich amplicon containing beads. In one embodiment of the enrichment process, the amplicon containing beads are retrieved by 5′-biotinlated oligos that are complementary to the 3′-end common sequence section of the amplicon. Then these can be extracted using streptavidin-coated magnetic beads. In another embodiment, the amplicon containing beads are hybridized with fluorophore labeled oligos that are complementary to the 3′-end common sequence section of the amplicon. Then the fluorophore-oligo bond beads are enriched by a flow cytometer.

The second part of the bead-based reactions (steps 1115 and 1118, FIG. 11) is the Sanger amplification reaction. These steps are designed to produce, on each bead, a full set of cleaned, fluorescence-labeled Sanger sequence fragments that are originated from one sequence template. An emulsion may be formed between a Sanger mix solution containing the beads from the first emPCR (1114 of FIG. 11) and mineral oil. The Sanger mix solution contains polymerase 1104, dNTP 1104, and fluorescence labeled ddNTP terminators 1109, and a primer 1108 (step 1115 of FIG. 11). Sanger amplification reaction is performed in a PCR tube on a PCR thermocycler. Commercially available kits (e.g. BigDye® Terminator v3.1 Cycle Sequencing Kits from ABI or DYEnamic ET mix from GE HealthCare) and protocols can be use for these steps. An annealing step at the end of a thermal cycling program can be used to bind the amplification products to the immobilized templates (step 1116 of FIG. D11). Emulsion breaking and bead washing conditions, such as using low temperature and high-salt buffer, can be utilized to ensure the retention of the Sanger amplification products on the beads (step 1117 of FIG. D11). The use of isopropanol or other solvent such as ethanol during emulsion breaking causes stronger binding between DNA molecules and is therefore okay for the process. The ability to perform on-bead purification to remove unused labeled terminators 1109 and other amplification reagents, which would interfere with signal detection and separation in electrophoresis, is a significant advantage of this method.

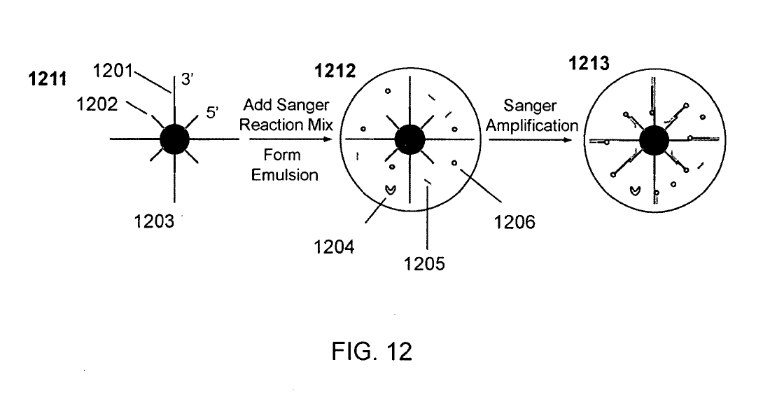

FIG. 12 shows an alternative bead surface composition compared with the one shown in FIG. 11. In this method a capture sequence 1202 is attached to the bead surface 1203 along with forward primers. The capture sequence 1202 is complementary to a part of 5′ common section of Sanger fragments and is designed to capture the Sanger fragments (1207 FIG. 12). In a preferred embodiment the sequence is located outside the section that is complementary to the above mentioned 5′-biotinylated oligo and therefore will not cause any problem to PCR beads enrichment. In a preferred embodiment, these capture sequences have free 5′ end to avoid any polymerase extension and to minimize steric effect for hybridization with Sanger fragments 1207. A potential advantage of mixing forward primers with capture sequences 1203 is that the reduced surface density of forward primers will likely improve the conditions for the formation of full-length first strand DNA 1201 in PCR reactions as well as primer extension in Sanger reactions.

Beads of various sizes, shapes, materials and porosities may be used in the methods of the present invention. Covalent attachment of oligo sequences, stabilities in emulsion PCR as well as Sanger reactions, surface loading densities, size distributions, and compatibility with gel electrophoresis are the factors to be considered during bead selection. Materials may include but are not limited to Sepharose® (GE Healthcare, former Amersham Biosciences) which is cross-linked agarose, cross-linked polyacrylamide (available from Thermo Scientific Pierce and other companies), TentaGel® (Rapp Polymere GmbH) which is polyethyleneglycol grafted on a low crosslinked polystyrene, and any other appropriate materials. Most beads are available with functional groups, such as N-hydroxysuccinimide ester (NHS) and amine, already on the bead surface and can be used for oligo attachment. In one embodiment, oligos containing either 3′ or 5′ terminal amine groups are attached to NHS functionalized beads by forming chemically stable amide bonds. In a preferred embodiment polyethylene glycol chains with 54 backbone atoms or longer are added to the surface attachment end of oligos for achieving reduced steric effect in polymerization as well hybridization reactions.

In a preferred embodiment, bead size is optimized by determined the necessary bead surface loading capacity and detection limit of capillary electrophoresis sequencing. Detection limit for laser induced fluorescence in capillary electrophoresis ranges from 10² to 10⁶ fluorophore molecules, depending on incident light intensity, fluorescence molecule, and detection optics. For capillary electrophoresis sequencing detection of 10⁵ fluorophores per band can readily achieved and 10 time reduction is possible (Blazej 2006). Therefore, for example, in order to read 600 bands 600×10⁵=6×10⁷=100 attomoles labeled Sanger fragments is needed and the number could be reduced to 10 attomoles.

### Electrophoresis Arrays

A second element of the device and methods of the present invention are high-density capillary array electrophoresis units. High-density capillary arrays to as opposed to the current discrete capillary tubes can be used to form a 3D electrophoresis separation system which will provide significantly increased throughput. The capillary arrays are available in various forms, sizes, and densities. The materials are made from glass processing technologies originally developed for optical fiber imaging applications. The arrays available from Scott are made either from clear or from high-contrast black glass materials. The internal diameter (or pore size) of the capillaries are between about 5 μm to about 1 mm. The lengths of the arrays are available from about 1 mm to about 2 m. The preferred arrays should contain densely packed and uniformly distributed capillary pores with smooth internal surfaces and polished at front and back end surfaces to an optical quality finish. In one embodiment a linear high-density capillary array from Schott is selected that has pore size of 50 82 m, capillary length of 80 cm, and packing number of 200,000 in a cross-section area of 20×20 mm². Other pore sizes, such as 5 μm, 10 μm, 20 μm, or 100 μm may be selected. Other packing numbers, such as 100, 1,000, 10,000, 1,000,000, or even higher, may be selected to fit specific applications.

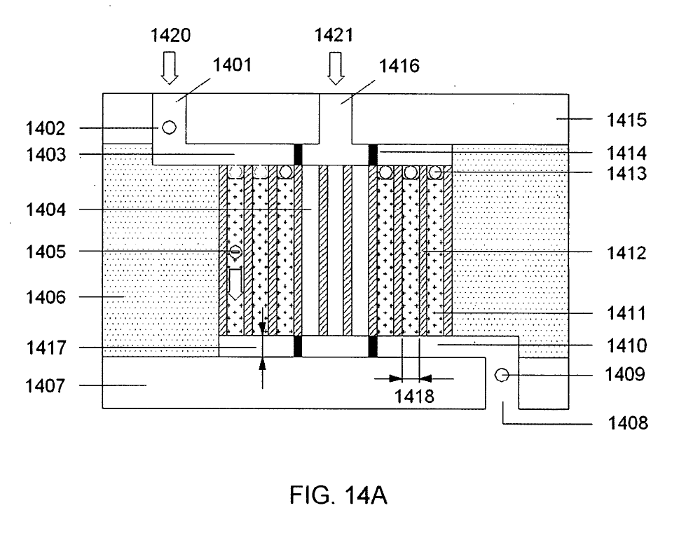

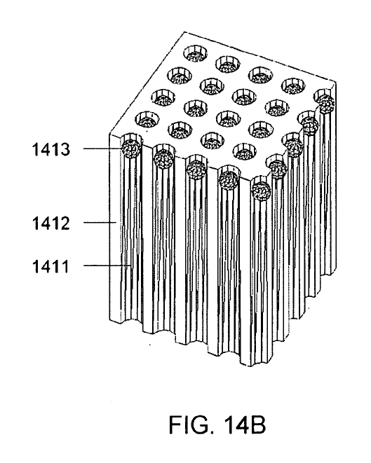

One embodiment electrophoresis cell containing a capillary array module is schematically shown in FIG. 14A. A capillary array module 1412 is placed at the center of the electrolyte cell 1406. FIG. 14B shows a 3D illustration of a capillary array module. While not shown in the figure, there are cooling and heating elements, temperature sensors, and temperature control mechanism build into the cell to either take away heat generated by Joule heating or add heat into the cell for maintaining desired temperatures for achieving optimized and reproducible electrophoresis separation. A heat-exchange zone 1404 shown in FIG. 14 utilizes a portion of the capillary tubes in the capillary array model. A heat exchange fluid, using for example water or air, is sent into the electrolyte cell through an entrance port 1416 to achieve an enhanced heat transfer. Cathode 1402 and anode 1409 electrodes are placed inside the electrolyte cell at suitable locations so that a uniform voltage drop will be produced across all capillaries. In a preferred embodiment the electrodes are made of platinum. Other electrode materials, such as porous carbon, may also be used. It should note that it is not necessary for the electrophoresis conditions of all the capillaries to be exactly the same nor it is necessary for the elapse times of specific sized fragments in all capillaries to be absolutely synchronized since gel run in each channel is independently analyzed, and thus one will be able to construct an individual chromatogram from each capillary based on the real-time fluorescence images that will discussed in a later section of this specification. There should also be considerations in the design of the probe and cell structure to allow the release of gas that is produced from the electrode surfaces. In a preferred embodiment, the electrolyte cell holder 1406 is made of glass, while other heat-resistant and non-conductive materials, such as heat-resistant plastic and ceramic, may also be used. The upper cap 1415 and the bottom window 1407 are made detachable for easy access to the capillary array during gel filling. In a preferred embodiment the cap is made of a heat-resistant and non-conductive material, such as polysulfone, polyphenylene sulfide, or ceramic. In a preferred embodiment, the bottom window 1407 is made of a thin glass, ranging from 100 μm to 500 μm. The gap 1417 between the glass window 1407 and the bottom surface of the capillary module 1412 should be relatively short, ranging from 50 μm to 200 μm. This allows a confocal laser scanning microscope, which will be described in a later section of this specification, to focus into the capillaries 1411. In a preferred embodiment, there a stead flow of electrolyte across destination chamber to flush away dye-labeled waste so as to minimize background level in photon detection signals. In addition to the electrolyte cell, an electrophoresis subsystem may also include a high-voltage power supply for running the electrophoresis and a PID temperature controller.

In a preferred embodiment, capillary surfaces are first treated with a chemical compound and then filled with gel. The method of surface treatment and gel formulations vary from one application to another and are well documented in literature such as the ones by Zhang 1999, Blazej 2006 and cited references which are incorporated herein by reference. In one embodiment of this invention, the filling of the capillary arrays is done by injection. An injection tool that has a gasket seal and a syringe is used.

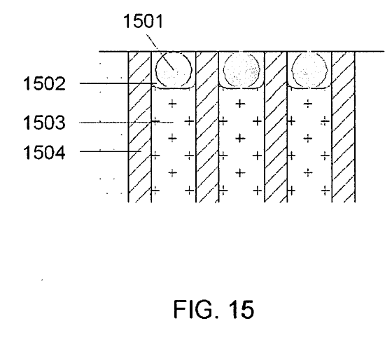

A number of techniques can be used to load beads into capillaries. In one embodiment, beads spread over a gel pad and are push into a capillary array block by gently pressing the array block surface against the gel pad. In another embodiment, shallow wells 1502 at the capillary inlet as shown in FIG. 15 are first created, flow a beads containing buffer solution to the surface, apply a gentle agitation and let the beads drop into wells by gravity (all cross-linked gel materials have a higher density than that of water). In a preferred embodiment, bead size should be slightly smaller than capillary pore size so that no well will be filled with two beads. In one embodiment, the wells 1501 are created during a gel filling process. Capillaries are filled from the bottom of a capillary array block with a slightly overfill, the extruded extra gel on top surface is wiped away with a squeeze, and then a small and fixed volume is pulled from the bottom to create the wells. In a preferred embodiment, one may pour a buffer solution on top of the array block before pulling to avoid air bubble trapping inside the wells. After the filling, a washing buffer solution is sent to the surface to wash away any extra beads.

Sharp sample injections of elution sequences are critical for obtaining high-resolution separation using capillary electrophoresis. In one aspect, one should take careful measures to prevent the dissociation of Sanger fragments from the beads during loading. This can be done by keeping the beads at low temperature (e.g. at 4° C.) and by using a non-denature buffer during the loading. Although not shown in FIG. 14, there are at least two liquid ports on electrolyte cell cap to allow fluid flow, buffer change, bead agitation, and extra-bead removal from the source chamber. After the completion of the bead loading, one may begin sample injection by turning on the electric field for the electrophoresis, replacing the non-denature buffer with an electrophoresis buffer, and apply a flash heating to the beads by turning on a rapid heater on the electrolyte cell cap (not shown in FIG. 14). The flash heating will cause the dissociation of Sanger fragments from the beads.

### Signal Detection

The third critical component of the proposed method is a fast confocal imager. The signal detection method used in current CE sequencers excites and collects fluorescence signals from side of one-dimensionally assembled capillaries. The method clearly cannot be used on the two-dimensionally assembled capillary arrays. We must use a method that is capable of collecting signals from all capillaries arranged in a two-dimensional plane. We choose confocal laser scanning imager because it is cable of detect signals from a very thin layer of materials while limiting interference from the materials above as well as below the signal collecting plane (or focal plane).

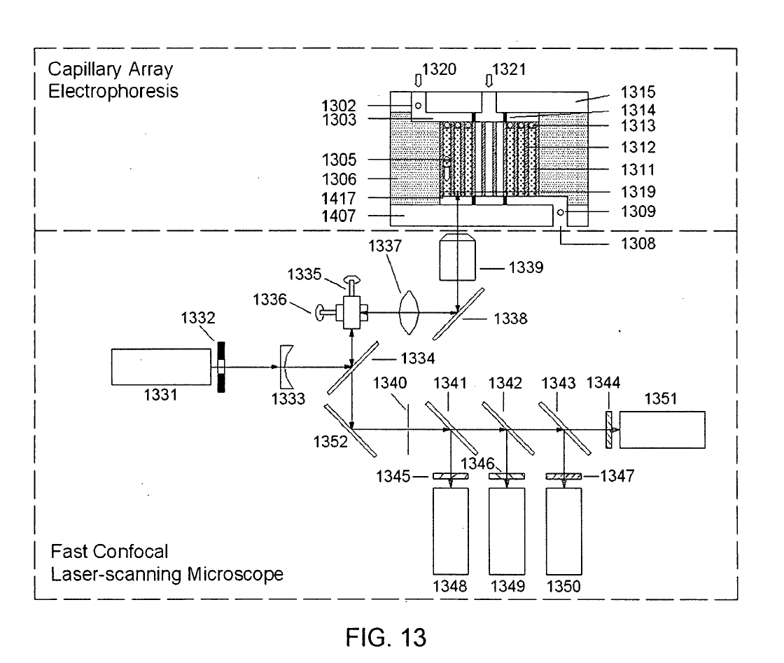

FIG. 13 shows a schematic diagram of a fast confocal laser scanning microscope subsystem in connection with an integrated system. This design is a modification to a video rate confocal microscope originally built in Parker's lab at UC Irvine (Callamaras 1999). A single dual line Ar ion laser 1331 is used for an excitation light source for four energy transfer dyes used in the labeled ddNTPs available from ABI and GE HearlthCare. During operation, a laser beam is expanded through a plano-concave lens 1333, is reflected in series by a dichroic filter 1334, Y galvanometer 1335, and X galvanometer 1336, passes through a microscope eyepiece 1337, is reflected by mirror 1338, and then passes a microscope objective lens 1339 and is focused to a focal plane 1319 above the lower surface of capillary array block 1312. The depth of confocal plane for the excitation can be adjusted by changing the focal length of the plano-concave lens 1333 or the distance between the plano-concave lens 1333 and the microscope objective lens 1339. X-Y scanning of the laser beam is performed by X and Y galvanometers 1335 and 1336. A high speed scanning at video frequency can be achieved by replacing one of the galvanometers with a resonant oscillator which operates at 8 kHz (available from General Scanning). Emission light from fluorophores is collected be the objective lens 1339, goes back in a reverse direction of the laser beam till hitting a dichroic filter 1334, which is a long-pass filter. The emission light passes through the dichroic filter 1334, is reflected by a mirror 1352, goes through an iris 1340, is selected by a set of dichroic filters (1341, 1342, and 1343) and bandpass filters (1345,1346, 1347, and 1344), and is detected by a photomultiplier (1348, 1349, 1350, and 1351) of a matching wavelength. The depth of confocal plane for the fluorescence detection can be adjusted by changing the aperture of the iris 1340.

As a high-throughput signal detector of the proposed capillary array electrophoresis an imager must meet several requirements. First, it must be fast enough to capture chromatograms at a sufficient resolution from all capillaries within a predefined scanning area. In general the time gap between two adjacent peaks of sequencing capillary electrophoresis is between 5 to 8 seconds.37 If 10 data points are required between the two adjacent peaks the imager scanning speed would have to be at least 2 frames per second. Second, the imager must have sufficient spatial resolutions in all three dimensions since the proposed capillary array is actually a 3D electrophoresis system with different sequence templates distributed in x-y directions and sequence fragments of different sizes distributed in z direction (which is the capillary axial direction). During Phase I project, we will use capillary array having a capillary diameter of 50 μm and a capillary center-to-center distance of 60 μm. Assuming a minimum requirement of capturing 5×5 pixels per capillary, the imager would need a resolution of 60/5=12 μm in x and y directions. In z direction, the distance between two adjacent peaks is about 1,500 μm at anode end of a capillary.37 To have truly resolved 10 data points between the two peaks a depth of focus must be no more than 1,500/10=150 μm. Based on previous result of a similar microscope design the above requirements are all achievable.38 As for image resolution, during Phase I plan to demonstrate 512×512 2.6×10⁵ pixels. At 2 frames per second, each PMT needs to be able to collect data at a rate of 2.6×10⁵×2=5.2×105 Hz. A type response time of PMTs is about 2 nano seconds which means a maximum data collection frequency of 1/(2×10⁻⁹)=5×108 Hz, which far exceeds our Phase I speed requirement and will provide us with a plenty of room for increasing data throughput during Phase II project. For example, we plan to demonstrate sequencing from 1 million capillaries during Phase II period. Assuming the same 5×5 pixels per capillary and 2 frames per second, we will need a data collection rate of 5×5×106×2=5×10⁷ Hz, which is still below the limit of PMTs. At system level, we recognize the challenge of making as well as a wide range of potential applications of fast and high resolution confocal imaging across an area as large as tens of cm². F

### System Integration and Operation

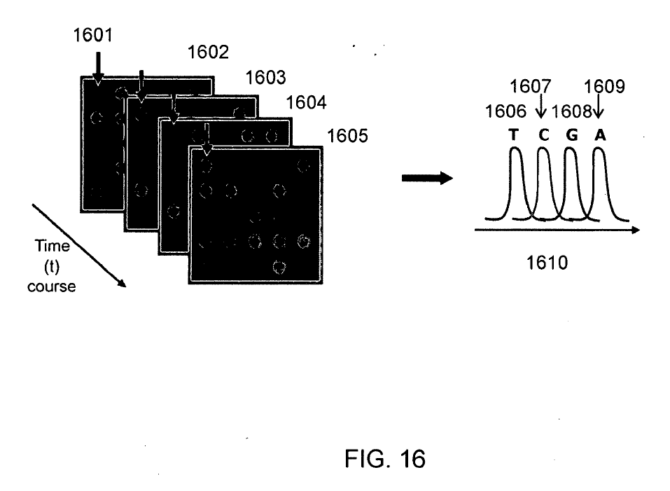

In addition to the components shown in FIG. 13, on need to add a fluid manifold, multi-zone temperature control unit, electronic drivers for galvanometers, electronic amplifiers for photomultipliers, and a computer system containing high-speed data acquisition boards and high-speed data storage. A software program is used to perform data collection and instrument controls. The program is able to locate capillary positions on images, extract signal intensities, and construct electropherograms by connecting the intensity data of all time points (FIG. 16).

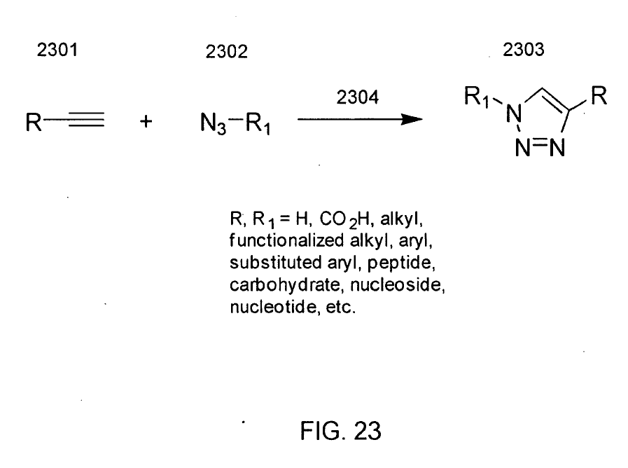

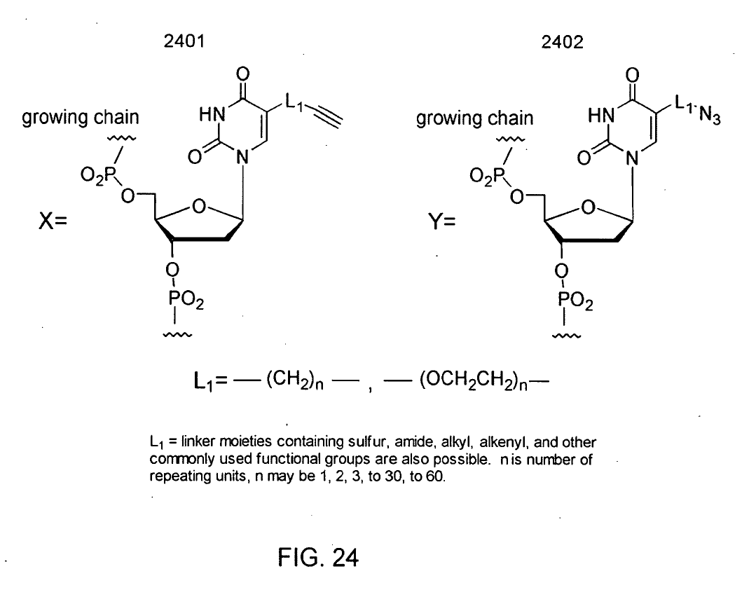

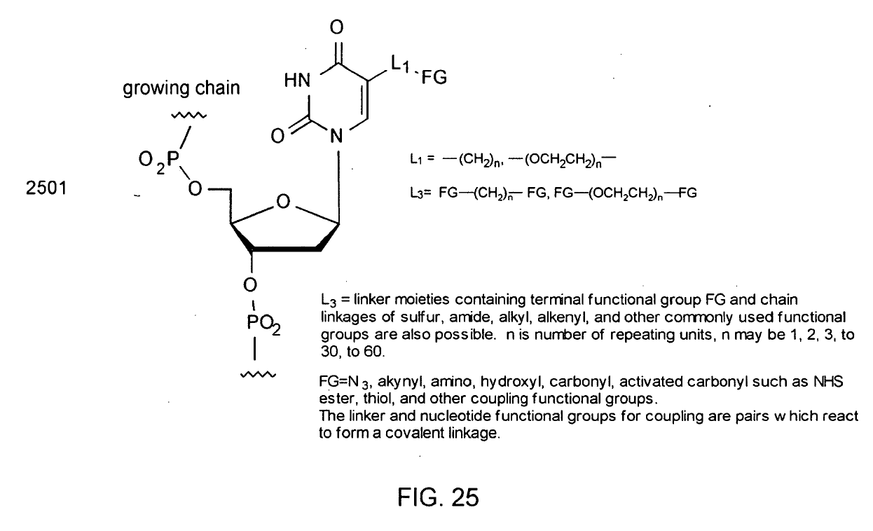

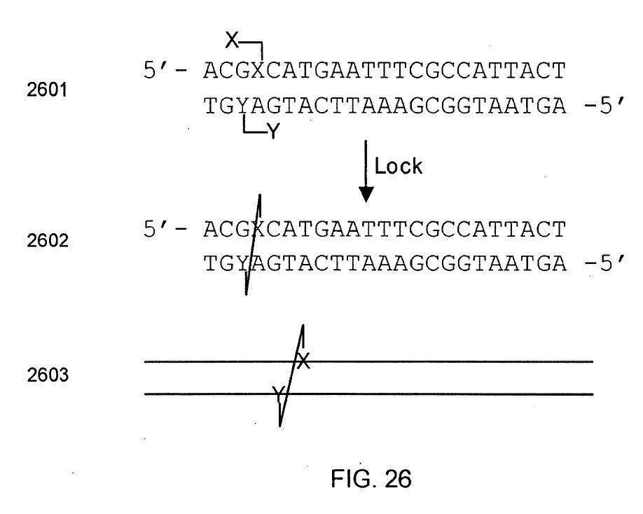

Described herein are also methods of making duplexes of nucleic acids which are locked once forming duplex (i.e. do not dissociate). Stable duplexes retains the solution molecules once they find the specific complementary sites and prevent surface molecules going back to the solution face. One method discloses the coupling reaction using the Huisgen cycloaddition reaction (click chemistry) (FIG. 23). The modified dU sites contain terminal alkyne groups (FIG. 24 and FIG. 25) and the linkers are of the suitable length for forming cross strand linkages in Click reaction or other conditions of coupling reactions. One embodiment has reaction in ethanol and water mixture and in the presence of CuSO₄ and Ph₃P for 2 hours in the presence of the sequences shown in FIG. 26.

### EXAMPLES

### Example 1

### Elution of Sequences in Capillary Bundle

A sequencing CE module was made from drawn glass to form a hollow channel bundle HOW MANY IN THE BUNDLE with 100 μm capillary inner diameter which had dimensions of 2×3 mm² at the channel cross section and was 5 cm in length. The sequencing channels were filled with 10% PAGE gel by capillary effect and the sample (described below) was loaded by applying the solution to half of the area of the bottom surface (which is perpendicular to the channels). A sample containing four fluorescence dye-labeled oligos of different lengths was used. The four oligos were FAM-18mer, Cy3-6mer, Cy3-38mer and FAM-46mer. The sequencing CE module was then placed in a horizontal electrophoresis apparatus for specified time (minutes), taken out to acquire images at the exit surface using an epifluorescence microscope (Olympus BX41 EPI fluorescence research microscope), and was placed back to the electrophoresis apparatus to continue the run. This process was repeated several times and the recorded images are shown in FIG. 5. The narrower slide of images in A and B (top row, FIG. C1) were control areas where there were no samples loaded. The time course is shown on top of the images. Tracking from 75-80 min, FAM-18mer was detected, followed by Cy3-6mer at 85 min, Cy3-38mer at 97 min, and finally FAM-46mer as a broad band centered around 102 min. It is noted that Cy3-6 oligo, GGTTGG, is a G-quadruplex motif; and the four stranded 6-mer behaved more like a 24-mer revealed by gel images. The Cy3-GGTTGG was studied in our lab for G-quadruplex formation under different conditions, confirming what we observed in the sequencing chip. By using two detection wavelengths, the top view images reflected what eluted from the capillary channels and the four oligonucleotides (FAM-18mer, Cy3-6mer, Cy3-38mer, and FAM-46mer) were resolved (images A and B, FIG. C1) in the run of an hour. The data are low resolution (the multiple channels which lighted up were mostly due to the cross in loading of the sample solution).

### U.S. Patent Documents

Gao, X., Zhou, X., and Gulari, E. “Method and apparatus for chemical and biochemical reactions using photogenerated reagents”. U.S. Pat. No. 6,426,184
Gao, X., Zhang, H., Yu, P., LeProust, E., Pellois, J. P. Xiang, Q., Zhou, X. “Linkers and co-coupling agents for optimization of oligonucleotide synthesis and purification on solid supports”. U.S. Pat. No. 7,211,654, AU2002305061.
Gao, X., Zhou, X., Cai, S. -Y, You, Q., Zhang, X. “Array oligomer synthesis and use” WO2004/039953.
Farooqui, F. and Reddy, P. M. (2003) Efficient synthesis of protein-oligonucleotide conjugates. US 2003/0092901.
Roullard, J. M., Gulari, E., Gao, X., Zhou, X. (2007) Method for forming molecular sequences on surfaces.

### Foreign Patent Documents

Gao, X., Zhou, X., and Gulari, E. “Method and apparatus for parallel synthesis of molecular sequence arrays using photo-generated reagents”. EP1054726B1, AU772531, CA2319587
Gao et al. PCT/US08/82167 Probe bead synthesis and use

### OTHER REFERENCES

Akeson, M., Branton, D., Kasianowicz, J. J., Brandin, E. and Deamer, D. W. (1999) Microsecond time-scale discrimination among polycytidylic acid, polyadenylic acid, and polyuridylic acid as homopolymers or as segments within single RNA molecules. Biophys J 77, 3227-33.
Albert, T. J., Molla, M. N., Muzny, D. M., Nazareth, L., Wheeler, D., Song, X., Richmond, T. A., Middle, C. M., Rodesch, M. J., Packard, C. J., Weinstock, G. M. and Gibbs, R. A. (2007) Direct selection of human genomic loci by microarray hybridization. Nat Methods 4, 903-905.
Backer, S. C. et al. (2005) “The External RNA Controls Consortium: a progress report” Nature Methods 2, 731-734.
Barski, A., Cuddapah, S., Cui, K., Roh, T. Y., Schones, D. E., Wang, Z., Wei, G., Chepelev, I. and Zhao, K. (2007) High-resolution profiling of histone methylations in the human genome. Cell 129, 823-837.
Bennett, S. T., Barnes, C., Cox, A., Davies, L. and Brown, C. (2005) Toward the 1,000 dollars human genome. Pharmacogenomics 6, 373-382.
Bentley, D. R. (2006) Whole-genome re-sequencing. Curr Opin Genet Dev 16, 545-552.
Bentley, D. R. et al. (2008) Accurate whole human genome sequencing using reversible terminator chemistry. Nature 456, 53-59.
Blazej, R. G., Kumaresan, P. and Mathies, R. A. (2006) Microfabricated bioprocessor for integrated nanoliterscale Sanger DNA sequencing. Proc Natl Acad Sci USA 103, 7240-7245.
Blazej, R. G., Kumaresan, P., Cronier, S. A. and Mathies, R. A. (2007) Inline injection microdevice for attomole-scale sanger DNA sequencing. Anal Chem 79, 4499-4506.
Branton, D. et al. (2008) The potential and challenges of nanopore sequencing. Nat Biotechnol 26, 1146-1153.
Buratti, E., Baralle, M., and Baralle, F. E., (2006) Defective splicing, disease and therapy: searching for master checkpoints in exon definition. NAR 34, 3494-510.
Callamaras, N. & Parker, I. (1999) Construction of a confocal microscope for real-time x-y and x-z imaging. Cell Calcium 26:271-280.
Carrilho, E., Ruiz-Martinez, M. C., Berka, J., Smirnov, I., Goetzinger, W., Miller, A. W., Brady, D. and Karger, B. L. (1996) Rapid DNA sequencing of more than 1000 bases per run by capillary electrophoresis using replaceable linear polyacrylamide solutions. Anal Chem 68, 3305-3313.
Cloonan, N., Forrest, A. R., Kolle, G., Gardiner, B. B., Faulkner, G. J., Brown, M. K., Taylor, D. F., Steptoe, A. L., Wani, S., Bethel, G., Robertson, A. J., Perkins, A. C., Bruce, S. J., Lee, C. C., Ranade, S. S., Peckham, H. E. Manning, J. M., McKernan, K. J. and Grimmond, S. M. (2008) Stem cell transcriptome profiling via massive-scale mRNA sequencing. Nat Methods 5, 613-619.
Connell, C. R. et al. (1987) Automated DNA Sequence Analysis. BioTechniques 5, 342-348.
Dahl, F. et al. (2007) Multigene amplification and massively parallel sequencing for cancer mutation discovery. Proc. Natl. Acad. Sci. USA 104, 9387-9392.
Dillmore, W. S., Yousaf, M. N. and Mrksich, M. (2004) A photochemical method for patterning the immobilization of ligands and cells to self-assembled monolayers. Langmuir 20, 7223-7231.
Dolnik, V., Liu, S. and Jovanovich, S. (2000) Capillary electrophoresis on microchip. Electrophoresis 21, 41-54.
Drmanac S, Kita D, Labat I, Hauser B, Schmidt C, Burczak J D, Drmanac R (1998) Accurate sequencing by hybridization for DNA diagnostics and individual genomics. Nat Biotechnol 16, 54-58.
Duke, T., Monnelly, G., Austin, R. H., Cox, E. C. (1997) Sequencing in nanofabricated arrays: a feasibility study. Electrophoresis 18, 17-22.
Eid, J. et al. (2008) Real-Time DNA sequencing from single polymerase molecules. Science. DOI: 10.1126/science.1162986.
Eriksson, J., Karamohamed, S. and Nyren, P. (2001) Method for real-time detection of inorganic pyrophosphatase activity. Anal Biochem 293, 67-70.
Ewing, B., and Green, P. (1998) Base-calling of automated sequencer traces using phred. II. Error probabilities. Genome Res. 8, 186-194.
Ewing, B., Hillier, L., Wendl, M. C., and Green, P. (1998) Base-calling of automated sequencer traces using phred. I. Accuracy assessment. Genome Res. 8, 175-185.
Gao, X., LeProust, E., Zhang, H., Srivannavit, O., Gulari, E., Yu, P., Nishiguchi, C., Xiang, Q., Zhou, X. (2001) Flexible DNA chip synthesis gated by deprotection using solution photogenerated acids. Nucleic Acids Res. 29, 4744-4750.
Greenleaf, W. J. and Block, S. M. (2006) Single-molecule, motion-based DNA sequencing using RNA polymerase. Science 313, 801.
http://cgap.nci.nih.gov/; http://cancergenome.nih.gov/
http://marketing.appliedbiosystems.com/images/Product/Solid_Knowledge/ABI- 5845_SOLID_Product_Spec_Sheet_loresfinal.pdf
http://www.bindingdb.org/bind/index.jsp
http://www.biotrove.com/what_we_do/openarray.html
http://www.illumina.com/downloads/GenomeAnalyzer_SpecSheet.pdf
http://www.lcsciences.com/products/genomics/oligomix/oligomix.html
http://www.lcsciences.com/products/genomics/specialty_genomics_arrays/specialty_genomics_microarrays.html
https://products.appliedbiosystems.com:443/ab/en/US/adirect/ab?cmd=catNavigate2&catID=60 1642&tab=TechSpec
Huse, S. M., Huber, J. A., Morrison, H. G., Sogin, M. L., and Welch, D. M. (2007) Accuracy and quality of massively-parallel DNA pyro-sequencing. Genome Biol. 8, R143.
International Human Genome Sequencing Consortium (2004) Finishing the euchromatic sequence of the human genome. Nature 431, 931-945.
Johnson et al. (2003) Genome-wide survey of human alternative pre-mRNA splicing with exon junction microarrays arrays detect alternative splicing in drug targets. Science 302, 2141-2144.
Ju J, Ruan C, Fuller C W, Glazer A N, Mathies R A (1995) Fluorescence energy transfer dye-labeled primers for DNA sequencing and analysis. Proc Natl Acad Sci USA 92:4347-4351.
Ju, J., Kim, D. H., Bi, L., Meng, Q., Bai, X., Li, Z., Li, X., Marma, M. S., Shi, S., Wu, J., Edwards, J. R., Romu, A. and Turro, N. J. (2006) Four-color DNA sequencing by synthesis using cleavable fluorescent nucleotide reversible terminators. Proc Natl Acad Sci USA 103, 19635-19640.
Kan C W, Doherty E A, Barron A E (2003) A novel thermogellingmatrix for microchannel DNA sequencing based on poly-N-alkoxyalkylacrylamide copolymers. Electrophoresis 24, 4161-4169.
Kasianowicz J. J., Brandin, E., Branton, D., and Deamer, D. W. (1996) Characterizaton of individual polynucleotide molecules using a membrane channel. Proc Natl Acad Sci USA 93, 13770-13773.
Kaushansky, K. (2007) The chronic myeloproliferative disorders and mutation of JAK2: Dameshek's 54 year old speculation comes of age. Best Pract Res Clin Haematol 20, 5-12.
Kozlov, I. A., Melnyk, P. C., Stromsborg, K. E., Chee, M. S., Barker, D. L. and Zhao, C. (2004) Efficient strategies for the conjugation of oligonucleotides to antibodies enabling highly sensitive protein detection. Biopolymers 73, 621-630.
Kumaresan, P., Yang, C. J., Cronier, S. A., Blazej, R. G. and Mathies, R. A. (2008) High-throughput single copy DNA amplification and cell analysis in engineered nanoliter droplets. Anal Chem 80, 3522-3529.
Lander, E. S. (2001) Initial sequencing and analysis of the human genome. Nature 409, 860-921.
Leamon, J. H., Braverman, M. S., and Rothberg, J. M. (2007) High-Throughput, massively parallel DNA sequencing technology for the era of personalized medicine. Gene Ther. Reg. 3, 15-31.
Leproust, E., Zhang, H., Yu, P., Zhou, X., and Gao, X. (2001) Characterization of oligodeoxyribonucleotide synthesis on glass plates. Nucleic Acids Res. 29, 2171-2180.
Levy, S., Sutton, G., Ng, P. C., Feuk, L., Halpern, A. L., Walenz, B. P., Axelrod, N., Huang, J., Kirkness, E. F., Denisov, G., Lin, Y., Macdonald, J. R., Pang, A. W., Shago, M., Stockwell, T. B., Tsiamouri, A., Bafna, V., Bansal V., Kravitz, S. A., Busam, D. A., Beeson, K. Y., McIntosh, T. C., Remington, K. A., Abril, J. F., Gill, J., Borman, J., Rogers, Y. H., Frazier, M. E., Scherer, S. W., Strausberg, R. L. and Venter, J. C. (2007) The diploid genome sequence of an individual human. PLoS Biol 5, e254.
Liu, C. N., Toriello, N. M. and Mathies, R. A. (2006) Multichannel PCR-CE microdevice for genetic analysis. Anal Chem 78, 5474-5479.
Liu, P., Seo, T. S., Beyor, N., Shin, K. J., Scherer, J. R. and Mathies, R. A. (2007) Integrated portable polymerase chain reaction-capillary electrophoresis microsystem for rapid forensic short tandem repeat typing. Anal Chem 79, 1881-1889.
Liu, S., Ren, H., Gao, Q., Roach, D. J., Loder, R. T., Jr., Armstrong, T. M., Mao, Q., Blaga, I., Barker, D. L. and Jovanovich, S. B. (2000) Automated parallel DNA sequencing on multiple channel microchips. Proc Natl Acad Sci USA 97, 5369-5374.
Makhijani, V., Roth, G. T., Gomes, X., Tartaro, K., Niazi, F., Turcotte, C. L., lrzyk, G. P., Lupski, J. R., Chinault, C., Song, X. Z., Liu, Y., Yuan, Y., Nazareth, L., Qin, X., Muzny, D. M., Margulies, M., Weinstock, G. M., Gibbs, R. A. and Rothberg, J. M. (2008) The complete genome of an individual by massively parallel DNA sequencing. Nature 452, 872-826.
Margulies et al. (2005) Genome sequencing in microfabricated high-density picolitre reactors. Nature 437, 376-380.
Maxam, A. M., and Gilbert, W. (1977) A new method for sequencing DNA. Proc. Natl. Acad. Sci. USA 74, 560-564.
Metzker, M. L. (2005) Emerging technologies in DNA sequencing. Genome Res 15, 1767-1776.
Mitra R. D., Shendure J., Olejnik J., Edyta-Krzymanska O., and Church G M (2003) Fluorescent in situ sequencing on polymerase colonies. Anal Biochem 320, 55-65.
Morozova, O. and Marra, M. A. (2008) Applications of next-generation sequencing technologies in functional genomics. Genomics 92, 255-264.
Mughal, T. I. and Goldman, J. M. (2007) Emerging strategies for the treatment of mutant Bcr-Abl T315I myeloid leukemia. Clin Lymphoma Myeloma 7 Suppl 2, S81-84.
Okou, D. T. et al. (2007) Microarray-based genomic selection for high-throughput resequencing. Nat. Methods, advance online publication 14 Oct. 2007 (doi:10.1038/nmeth1109).
Parameswran, P., Jalili, R., Tao, L., Shokralla, S., Gharizadeh, B., Ronaghi, M., and Fire, A. Z. (2007) A pyrosequecing-tailored nucleotide barcode design unveils opportunities for large-scale sample multiplexing. Nucleic Acids Res. Advance Access Oct. 11, 2007 doi:10.1093.
Porreca, G. J., Zhang, K., Li, J. B., Xie, B., Austin, D., Vassallo, S. L., LeProust, E. M., Peck, B. J., Emig, C. J., Dahl, F., Gao, Y., Church, G. M. and Shendure, J. (2007) Multiplex amplification of large sets of human exons. Nat Methods 4, 931-936.
Prober J. M., Trainor G. L., Dam R. J., Hobbs F. W., Robertson C. W., Zagursky R. J., Cocuzza A. J., Jensen M. A., and Baumeister, K. (1987) A system for rapid DNA sequencing with fluorescent chain-terminating dideoxynucleotides. Science 238, 36-341.
Raddatz, S., Mueller-lbeler, J., Kluge, J., Wass, L., Burdinski, G., Havens, J. R., Onofrey, T. J., Wang, D., and Schweitzer, M. (2002) Hydrazide oligonucleotides: new chemical modification for chip array attachment and conjugation. Nucleic Acids Res., 30, 4793-4802.
Reviewed in Gao, X., Gulari, E., and Zhou, X. (2004) In situ synthesis of oligonucleotide microarrays. Biopolymers 73, 579-596.
Reviewed in Gao, X., Gulari, E., and Zhou, X. (2004) In situ synthesis of oligonucleotide microarrays. Biopolymers 73, 579-596.
Reviewed in Gao, X., Pellois, J. P., Kim, K., Na, Y., Gulari, E., and Zhou, X. (2004) High density peptide microarrays. In situ synthesis and applications. Molecular Diversity 8, 177-187.
Ronaghi, M., Karamohamed, S., Pettersson, B., Uhlen, M. and Nyren, P. (1996) Real-time DNA sequencing using detection of pyrophosphate release. Anal Biochem 242, 84-89.
Ronaghi, M., Uhlen, M. and Nyren, P. (1998) A sequencing method based on real-time pyrophosphate. Science 281, 363-365.
Sanger, F., Air, G. M., Barrell, B. G. Brown, N. L., Coulson, A. R., Fiddes, C. A., Hutchison, C. A., Slocombe, P. M., Smith, M. (1977) Nucleotide sequence of bacteriophage phi X174 DNA. Nature 265, 687-695.
Sanger, F., Coulson, A. R. (1975) A rapid method for determining sequences in DNA by primed synthesis with DNA polymerase. J. Mol. Biol. 94, 441-448; (b) Sanger, F;, Nicklen, S., Coulson, A. R. (1977) DNA sequencing with chainterminating inhibitors, Proc. Natl. Acad. Sci. USA 74, 5463-5467.
Shendure, J., Mitra, R. D., Varma, C., and Church, G. M. (2004) Advanced sequencing technologies: methods and goals. Nat Rev Genet 5, 335-344.
Shendure, J., Porreca G. J., Reppas N. B., Lin X., McCutcheon J. P., Rosenbaum A. M., Wang M. D., Zhang K., Mitra R. D., and Church G. M. (2005) Accurate multiplex polony sequencing of an evolved bacterial genome. Science 309, 1728-1732.
Smith L. M., Sanders J. Z., Kaiser R. J., Hughes P., Dodd C., Connell C. R., Heiner C., Kent S. B., and Hood L E (1986) Fluorescence detection in automated DNA sequence analysis. Nature 321, 674-679.
Soellner, M. B., Dickson, K. A., Nilsson, B. L., and Raines, R. T. (2003) Site-specific protein immobilization by Staudinger ligation. J. Am. Chem. Soc. 125, 11790-11791.
Soni, G. V. and Meller, A. (2007) Progress toward Ultrafast DNA Sequencing Using Solid-State Nanopores. Clin Chem 53, 1996-2001.
Srivannavit, O., Gulari, M., Gulari, E., LeProust, E., Pellois, J. P., Gao, X., and Zhou, X. (2004) Design and fabrication of microwell array chips for a solution-based, photogenerated acid-catalyzed parallel oligonuclotide DNA synthesis. Sensors and Actuators A. 116, 150-160.
Sundquist, A., Ronaghi, M., Tang, H., Pevzner, P. and Batzoglou, S. (2007) Whole-genome sequencing and assembly with high-throughput, short-read technologies. PLoS ONE 2, e484.
Szekely, M. (1977) Sequencing DNA. Nature 267, 104.
The Gene Ontology (GO) project (http://www.geneontology.org/); Alternative Exon Database (http://www.ebi.ac.uk/asd/aedb/)

These are listed at www.lcsciences.com.
Tian, J., Gong, H., Sheng, N., Zhou, X., Gulari, E., Gao, X., and Church, G. (2004) Accurate multiplex gene synthesis from programmable DNA chips. Nature 432, 1050-1054.
Venter J C, et al. (2001) The sequence of the human genome. Science 291, 1304-1351.
Wang, Q., Chan, T. R., Hilgraf, R., Fokin, V. V., Sharpless, K. B., and Finn, M. G. (2003) Bioconjugation by copper(I)-catalyzed azide-alkyne [3+2] Cycloaddition. J. Am. Chem. Soc., 125, 3192-3193.
Warnecke, F. and Hugenholtz, P. (2007) Building on basic metagenomics with complementary technologies. Genome Biol 8, 231.
Weisberg, E., Manley, P. W., Cowan-Jacob, S. W., Hochhaus, A. and Griffin, J. D. (2007) Second generation inhibitors of BCR-ABL for the treatment of imatinib-resistant chronic myeloid leukaemia. Nat Rev Cancer 7, 345-356.
Wheeler, D. A., Srinivasan, M., Egholm, M., Shen, Y., Chen, L., McGuire, A., He, W., Chen, Y. J., Wold, B. and Myers, R. M. (2008) Sequence census methods for functional genomics. Nat Methods 5, 19-21.
Zhang, J., Voss, K. O., Shaw, D. F., Roos, K. P., Lewis, D. F., Yan, J., Jiang, R., Ren, H., Hou, J. Y., Fang, Y., Puyang, X., Ahmadzadeh, H., and Dovichi, N. J. (1999) A multiple-capillary electrophoresis system for small-scale DNA sequencing and analysis. Nucleic Acids Res. 27, e36.
Zhou, H., Miller, A. W., Sosic, Z., Buchholz, B., Barron, A. E., Kotler, L. and Karger, B. L. (2000) DNA sequencing up to 1300 bases in two hours by capillary electrophoresis with mixed replaceable linear polyacrylamide solutions. Anal Chem 72, 1045-1052.
Zhou, X., Cai, S., Hong, A., Yu, P., Sheng, N., Srivannavit, O., Yong, Q., Muranjan, S., Rouilard, J. M., Xia, Y., Zhang, X., Xiang, Q., Ganesh, R., Zhu, Q., Makejko, A., Gulari, E., and Gao, X. (2004) Microfluidic PicoArray synthesis of oligodeoxynucleotides and simultaneously assembling of multiple DNA sequences. Nucleic Acids Res. 32, 5409-5417.
Perez-Balderas. F., etc. (2003) Multivalent neoglycoconjugates by regiospecific cycloaddition of alkynes and azides using organicsoluble copper catalysts. Org. Lett. 5, 1951-1954.

1-40. (canceled)

41. A method for determining the nucleotide base sequence of a DNA molecule, comprising:
a) mixing a plurality of nucleic acid templates and beads comprising oligonucleotides attached to the bead capable of binding the nucleic acid templates to the beads in a first reaction solution comprising reagents necessary to amplify the nucleic acid templates to form an amplification mixture;
b) forming a first emulsion from the amplification mixture so as to create a plurality of droplets comprising the nucleic acid templates, beads, and first reaction solution, wherein at least one of the droplets comprises a single nucleic acid template and a single bead encapsulated in the first reaction solution, wherein the droplets are contained in the same vessel;
c) amplifying the nucleic acid templates in the droplets to form amplified copies of the nucleic acid templates;
d) breaking the first emulsion and washing the beads;
e) mixing the nucleic acid templates attached to the beads in a second reaction solution comprising four different deoxynucleoside triphosphates, a processive DNA polymerase, and four different labeled DNA synthesis terminating agents which terminate DNA synthesis at a specific nucleotide base;
f) forming an second emulsion to create a plurality of droplets comprising the nucleic acid templates, beads, and second reaction solution, wherein at least one of the droplets comprises DNA templates of a single sequence on a single bead encapsulated in the second reaction solution, wherein the droplets are contained in the same vessel and wherein each termination agent terminates DNA synthesis at a different nucleotide base, thereby forming terminated sequences;
g) breaking the second emulsion and washing the beads while retaining the terminated sequences on the beads;
h) loading the beads into a plurality of capillaries such that at least one of the capillaries contains a single bead;
i) dissociating the terminated sequences from the bead;
j) separating the terminated sequences according to their size; and
k) detecting the terminated sequences by the labeled synthesis terminating reagents whereby at least a part of the nucleotide base sequence of said DNA molecule can be determined.

42. The method of claim 41 wherein the nucleic acid templates are from 25-1500 bases in length.

43. The method of claim 41 wherein the oligonucleotide attached to the bead is a primer molecule or a capture molecule.

44. The method of claim 41 further comprising incubating the beads such that the nucleic acid template is single stranded prior to step (e) and after step (d).

45. The method of claim 41 wherein the DNA synthesis terminating agents are dideoxynucleotides labeled with fluorescent dyes.

46. The method of claim 45 wherein the dideoxynucleotides are ddT, ddA, ddG and ddC.

47. The method of claim 41, 45 or 46 wherein the terminated sequences are detected by a confocal microscope.

48. The method of claim 41 wherein the terminated sequences are dissociated from the bead by heat.

49. The method of claim 41 wherein the plurality of capillaries is selected from the group consisting of 100, 101-1,000, 1,001-10,000, 10,001-100,000, 100,001-1,000,000, and 1,000,001-10,000,000.

50. The method of claim 41 wherein the capillaries are monolithic structures.

51. A method for preparing labeled terminated DNA sequences comprising:
a) providing a plurality of beads comprising a plurality of DNA templates of a single sequence on each bead;
b) mixing the nucleic acid templates attached to the beads in a reaction solution comprising four different deoxynucleoside triphosphates, a processive DNA polymerase, and four different labeled DNA synthesis terminating agents which terminate DNA synthesis at a specific nucleotide base;
c) forming an emulsion to create a plurality of droplets comprising the nucleic acid templates, beads, and the reaction solution, wherein at least one of the microreactors comprises DNA templates of a single sequence on a single bead encapsulated in the reaction solution, wherein the droplets are contained in the same vessel and wherein each termination agent terminates DNA synthesis at a different nucleotide base, thereby forming terminated sequences.

52. The method of claim 51 wherein the plurality of beads is greater than 1,000,000.

53. The method of claim 51 wherein the plurality of beads is between about 10,000 and 10,000,000.

54. A device for detecting fluorescently labeled terminated DNA sequences comprising:
a) a plurality of capillary tubes filled with a gel matrix;
b) a mechanism for introducing a single bead comprising fluorescently labeled terminated DNA sequences into each capillary tube;
c) a mechanism for dissociating the fluorescently labeled terminated DNA sequences from the bead; and
d) a signal detector for detecting the fluorescently labeled terminated DNA sequences

55. The device of clam 54 wherein the plurality of capillary tubes is a capillary block.

56. The device of claim 54 wherein the capillary block comprises more that 1,000 capillary tubes.

57. The device of claim 54 wherein the capillary block comprises from about 1,000 to about 1,000,000 capillary tubes.

58. The device of claim 14 further comprising an electrolyte cell holder.

59. The device of claim 14 further comprising a heat transfer device.

60. The device of clam 14 further comprising a cap.

61. The device of claim 14 wherein the signal detector is a confocal scanner.

62. The device of claim 21 wherein the confocal scanner comprises a laser.
